(19) United States
(12) Patent Application Publication
(10) Pub. No.: US 2025/0377363 A1
(43) Pub. Date: Dec. 11, 2025

(54) METHODS RELATING TO DIAGNOSING STABILITY FOLLOWING RENAL TRANSPLANTATION

(71) Applicants: THE CHILDREN'S MEDICAL CENTER CORPORATION, Boston, MA (US); THE UAB RESEARCH FOUNDATION, Birmingham, AL (US); THE UNIVERSITY OF BRITISH COLUMBIA, Vancouver (CA)

(72) Inventors: David M. BRISCOE, Sharon, MA (US); Tom David BLYDT-HANSEN, North Vancouver (CA); Michael Edward SEIFERT, Birmingham, AL (US); Alvin Thong Juak KHO, Boston, MA (US)

(73) Assignees: THE CHILDREN'S MEDICAL CENTER CORPORATION, Boston, MA (US); THE UAB RESEARCH FOUNDATION, Birmingham, AL (US); THE UNIVERSITY OF BRITISH COLUMBIA, Vancouver (CA)

(21) Appl. No.: 18/869,619

(22) PCT Filed: Jun. 1, 2023
(86) PCT No.: PCT/US2023/024149

§ 371 Date: Nov. 26, 2024

Related U.S. Application Data
(60) Provisional application No. 63/348,558, filed on Jun. 3, 2022.
(60) Provisional application No. 63/438,087, filed on Jan. 10, 2023.

(51) Int. Cl. G01N 33/68

(52) U.S. Cl. CPC G01N 33/6863; G01N 33/6893; G01N 2333/521; G01N 2800/245

(57) ABSTRACT

Clinical detection or diagnosis of transplant rejection currently utilizes various methods that rely on qualitative rather than quantitative data, or quantitative data that has low sensitivity and/or has a high rate of operator error. Provided herein are methods for identifying kidney transplant rejection in patents that is automated and consistent for the user. Further described herein are cut-offs for transplant biomarkers that relate to renal transplantation stability or rejection.

### CROSS REFERENCE TO RELATED APPLICATIONS

This application claims benefit under 35 U.S.C. § 119 (e) of U.S. Provisional Application No. 63/348,558, filed Jun. 3, 2022, and U.S. Provisional Application No. 63/438,087, filed Jan. 10, 2023, the contents each of which are incorporated herein by reference in their entireties.

### TECHNICAL FIELD

The technology described herein relates to methods for detecting and/or treating transplant rejection, particularly kidney transplant rejection.

### BACKGROUND

Clinical detection or diagnosis of transplant rejection currently utilizes various methods that rely on qualitative rather than quantitative data, or quantitative data that has low sensitivity and/or has a high rate of operator error. This type of data can result in detecting rejection of organs after the optimum time for treatment/intervention, an increased likelihood that the organ will be fully rejected, and additional complications. Late detection of transplant rejection also results in not being able to effectively use rejection therapy and/or immunosuppressive therapy to mitigate the effects of transplant rejection. Described herein are assays that can provide automation and/or avoidance of operator error. Further described herein are cut-offs for transplant biomarkers that relate to renal transplantation stability or rejection.

### SUMMARY

The technology as described herein relates to methods of identifying patients who are at risk for kidney transplant rejection using an automated system that detects the level of certain proteins in a patient sample. Such automated systems can include a cartridge pre-loaded with detection reagents. If the level of the proteins is outside of the range specified, the patient would be considered to be at increased risk of kidney transplant rejection and/or to require treatment for transplant rejection.

In one aspect of any of the embodiments, described herein is a method of identifying a kidney transplant patient as being stable and not in transplant rejection, the method comprising:
a) contacting a urine sample with antibodies specific for each of the CXCL9, CXCL10, CCL2, and VEGF-A proteins;
b) detecting the amount of each of CXCL9, CXCL10, CCL2, and VEGF-A bound to the antibodies, providing a data set comprising values for the level of each of CXCL9, CXCL10, CCL2, and VEGF-A detected;
c) analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant;
   wherein the weighting parameters are, 0.73797 for CXCL9, −0.046218 for CXCL10, 0.020161 for CCL2, and −0.21309 for VEGF-A and the constant is −4.0624; thereby providing a score; and
d) identifying a kidney transplant patient as being stable and not in transplant rejection based on the score being below a predetermined threshold.

In one aspect of any of the embodiments, described herein is a method of identifying a kidney transplant patient as being stable and not in transplant rejection, the method comprising:
a) contacting a urine sample with antibodies specific for each of CXCL9, CXCL10, CCL2, and VEGF-A proteins;
b) detecting the amount of each of CXCL9, CXCL10, CCL2, and VEGF-A bound to the antibodies, providing a data set comprising values for the level of each of CXCL9, CXCL10, CCL2, and VEGF-A detected;
c) analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant; and
   the data set further comprises a value for the time from transplant of the kidney and the weighting process further comprises applying a weighting parameter for the time from transplant of the kidney,
   wherein the weighting parameters are 0.75542 for CXCL9, 0.057341 for CXCL10, 0.095251 for CCL2, −0.34348 for VEGF-A, and 0.71722 for time from transplant; and
   wherein the constant is −7.1573 thereby providing a score; and
d) identifying a kidney transplant patient as being stable and not in transplant rejection based on the score being below a predetermined threshold.

In some embodiments of any of the aspects, the contacting and detecting steps are performed in an automated device.

In some embodiments of any of the aspects, the automated device comprises a cartridge comprising the antibodies.

In some embodiments of any of the aspects, the detecting step comprises simultaneously detecting the amount of each of CXCL9, CXCL10, CCL2, and VEGF-A bound to the antibodies.

In some embodiments of any of the aspects, the urinary protein levels are normalized to urinary creatinine.

In one aspect of any of the embodiments, described herein is a method of treating kidney transplant rejection in a patient who has received a kidney transplant, the method comprising:
a) administering rejection therapy to a patient determined to have:
   i) a urinary protein level of CXCL9 greater than 78.832 pg/mL;
   ii) a urinary protein level of CXCL10 greater than 8.5748 pg/mL;
   iii) a urinary protein level of CCL2 greater than 205.67 pg/mL; and
   iv) a urinary protein level of VEF-A greater than 142.98 pg/mL; and
b) administering standard immunosuppression therapy to a patient determined not to have:
   i) a urinary protein level of CXCL9 greater than 78.832 pg/mL;
   ii) a urinary protein level of CXCL10 greater than 8.5748 pg/mL;
   iii) a urinary protein level of CCL2 greater than 205.67 pg/mL; and
   iv) a urinary protein level of VEF-A greater than 142.98 pg/mL.

In one aspect of any of the embodiments, described herein is a method of treating kidney transplant rejection in a patient who has received a kidney transplant, the method comprising:
a) administering rejection therapy to a patient determined to have:
   i) a urinary protein level of CXCL9 greater than a normal or borderline level in Table 1;
   ii) a urinary protein level of CXCL10 greater than a normal or borderline level in Table 1;
   iii) a urinary protein level of CCL2 greater than a normal or borderline level in Table 1; and
   iv) a urinary protein level of VEF-A greater than a normal or borderline level in Table 1; and
b) administering standard immunosuppression therapy to a patient determined not to have:
   i) a urinary protein level of CXCL9 greater than a normal or borderline level in Table 1;
   ii) a urinary protein level of CXCL10 greater than a normal or borderline level in Table 1;
   iii) a urinary protein level of CCL2 greater than a normal or borderline level in Table 1; and
   iv) a urinary protein level of VEF-A greater than a normal or borderline level in Table 1.

In some embodiments of any of the aspects, the normal or borderline level in Table 1 is the 75% tile normal or borderline level.

In some embodiments of any of the aspects, the rejection therapy comprises a test to identify disease in the graft, a renal biopsy, methylprednisolone, plasma exchange, intravenous immunoglobulin, anti-thymocyte globulin, anti-CD20 antibody, lymphocyte-depleting antibody, or a combination thereof.

In some embodiments of any of the aspects, the rejection therapy comprises methylprednisolone, plasma exchange, intravenous immunoglobulin, anti-thymocyte globulin, anti-CD20 antibody, lymphocyte-depleting antibody, or a combination thereof.

In some embodiments of any of the aspects, the anti-CD20 antibody is rituximab, ocrelizumab, ofatumumab, or obinutuzumab.

In some embodiments of any of the aspects, the lymphocyte-depleting antibody is muromonab.

In some embodiments of any of the aspects, the standard immunosuppression therapy comprises a reduction or cessation of a corticosteroid, calcineurin inhibitor, mTOR inhibitor, belatacept, everolimus, prednisone, tacrolimus, mycophenolate, prednisolone, cyclosporine, siroliums, or azathioprine; or an avoidance of a further test or biopsy.

In some embodiments of any of the aspects, the standard immunosuppression therapy comprises corticosteroids, calcineurin inhibitors, mTOR inhibitors, or a combination thereof.

In some embodiments of any of the aspects, the standard immunosuppression therapy comprises belatacept, everolimus, prednisone, tacrolimus, mycophenolate, prednisolone, cyclosporine, siroliums, azathioprine, or a combination thereof.

In one aspect of any of the embodiments, described herein is a method of detecting kidney tissue, optionally kidney tissue from a kidney transplant, undergoing pathological apoptosis, the method comprising:
a) contacting a urine sample obtained from the kidney tissue with antibodies specific for each of CXCL9, CXCL10, CCL2, and VEGF-A proteins;
b) detecting the amount of each of CXCL9, CXCL10, CCL2, and VEGF-A bound to the antibodies, providing a data set comprising values for the level of each of CXCL9, CXCL10, CCL2, and VEGF-A detected.

In some embodiments of any of the aspects, the detecting further comprises analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations.

In some embodiments of any of the aspects, the weighting parameters are, 0.73797 for CXCL9, −0.046218 for CXCL10, 0.020161 for CCL2, and −0.21309 for VEGF-A.

In some embodiments of any of the aspects, the logistical regression module further comprises applying a constant.

In some embodiments of any of the aspects, the constant is −4.0624.

In some embodiments of any of the aspects, the data set further comprises a value for the time from transplant of the kidney.

In some embodiments of any of the aspects, the weighting process further comprises applying a weighting parameter for the time from transplant of the kidney.

In some embodiments of any of the aspects, the weighting parameters are 0.75542 for CXCL9, 0.057341 for CXCL10, 0.095251 for CCL2, −0.34348 for VEGF-A, and 0.71722 for time from transplant.

In some embodiments of any of the aspects, the weighting process further comprises applying a constant.

In some embodiments of any of the aspects, the constant is −7.1573.

In some embodiments of any of the aspects, the method further comprises:
a) determining a score based on the urinary protein levels; and
b) identifying the kidney tissue as undergoing apoptosis based on the score being above a predetermined threshold.

In some embodiments of any of the aspects, the method further comprises identifying whether the kidney tissue is undergoing apoptosis and outputting said identification on a display.

In some embodiments of any of the aspects, the urine sample is obtained from a patient and the method further comprises assigning the patient to a rejection category according to the score and outputting the rejection category on a display.

In some embodiments of any of the aspects, the urinary protein levels are normalized to urinary creatinine.

In some embodiments of any of the aspects, the patient is a pediatric patient.

In one aspect of any of the embodiments, described herein is a method of treating kidney transplant rejection in a patient who has received a kidney transplant, the method comprising:
determining a urinary protein level of CXCL9; a urinary protein level of CXCL10; a urinary protein level of CCL2; and a urinary protein level of VEGF-A of a urine sample obtained from the kidney transplant;
predicting whether the patient will experience an acute rejection based on an acute rejection prediction score output from a logistic regression model, wherein the urinary protein level of CXCL9; the urinary protein level of CXCL10; the urinary protein level of CCL2; and the urinary protein level of VEF-A measured from the patient are input into the logistic regression model;
administering rejection therapy to the patient if the acute rejection prediction score exceeds a predetermined threshold; and
administering standard immunosuppression therapy to the patient if the acute rejection prediction score does not exceed a predetermined threshold.

In some embodiments of any of the aspects, the method further comprises determining a time after the transplant is received by the patient, and wherein the time after the transplant is input into the logistic regression model.

In some embodiments of any of the aspects, the logistic regression model includes a preliminary technical control step checking that the determined CXCL9 and CXCL10 levels are correlated to within a predetermined standard error (S.E.) value of a training set linear regression fit from one another with a predetermined slope and a predetermined y-intercept value.

In some embodiments of any of the aspects, the logistic regression model includes a −4.0624 constant 1, a weighting value of −0.020161 for the CCL2 level, a weighting value of −0.046218 for the CXCL10 level, a weighting value of 0.73797 for the CXCL9 level, and a weighting value of −0.21309 for the VEGF-A level.

In some embodiments of any of the aspects, the logistic regression model includes a −7.1573 constant 1, a weighting value of 0.095251 for the CCL2 level, a weighting value of 0.057341 for the CXCL10 level, a weighting value of 0.75542 for the CXCL9 level, a weighting value of −0.34348 for the VEGF-A level, and a weighting value of 0.71722 for the time after the transplant.

In some embodiments of any of the aspects, the urinary protein level of CXCL9; the urinary protein level of CXCL10; the urinary protein level of CCL2; the urinary protein level of VEF-A; and time from transplant are modified by adding a +1 pseudocount unit and by log 2-transforming the resulting values.

In some embodiments of any of the aspects, the contacting and/or detecting steps are performed in an automated device.

In some embodiments of any of the aspects, the automated device comprises a cartridge comprising the antibodies.

In some embodiments of any of the aspects, the detecting step comprises simultaneously detecting the amount of each of CXCL9, CXCL10, CCL2, and VEGF-A bound to the antibodies.

In some embodiments of any of the aspects, the urinary protein levels are normalized to urinary creatinine.

In one aspect of any of the embodiments, described herein is a cartridge comprising antibodies specific for each of the CXCL9, CXCL10, CCL2, and VEGF-A proteins, for use in the method of any of embodiments described herein.

In one aspect of any of the embodiments, described herein is a method of diagnosing kidney rejection in a subject who has received a kidney transplant, the method comprising, a) measuring the combination of: a urinary protein level of CXCL9; a urinary protein level of CXCL10; a urinary protein level of CCL2; and a urinary protein level of VEGF-A in a urine sample obtained from the subject; wherein the measuring is performed using a BIOTECHNE cartridge comprising detection antibodies for CXCL9, CXCL10, CCL2, and VEGF-A; and b) determining an acute rejection prediction score output from a logistic regression model using the levels measured in step a); and determining the subject has kidney rejection the acute rejection prediction score exceeds a predetermined threshold.

In some embodiments of any of the aspects, the logistic regression model further comprises the time elapsed from the kidney transplant.

In some embodiments of any of the aspects, the logistic regression model includes a preliminary technical control step checking that the determined CXCL9 and CXCL10 levels are correlated to within a predetermined standard error (S.E.) value of a training set linear regression fit from one another with a predetermined slope and a predetermined y-intercept value.

In some embodiments of any of the aspects, the logistic regression model includes a −4.0624 constant 1, a weighting value of −0.020161 for the CCL2 level, a weighting value of −0.046218 for the CXCL10 level, a weighting value of 0.73797 for the CXCL9 level, and a weighting value of −0.21309 for the VEGF-A level.

In some embodiments of any of the aspects, the logistic regression model includes a −7.1573 constant 1, a weighting value of 0.095251 for the CCL2 level, a weighting value of 0.057341 for the CXCL10 level, a weighting value of 0.75542 for the CXCL9 level, a weighting value of −0.34348 for the VEGF-A level, and a weighting value of 0.71722 for the time after the transplant.

In some embodiments of any of the aspects, the urinary protein level of CXCL9; the urinary protein level of CXCL10; the urinary protein level of CCL2; the urinary protein level of VEGF-A; and time from transplant are modified by adding a +1 pseudocount unit and by log 2-transforming the resulting values.

In some embodiments of any of the aspects, the measuring is performed in an ELLA device.

### DETAILED DESCRIPTION

As described herein, the inventors have found that specific assays (e.g., multiplexed assays) for renal transplant rejection provide a strikingly high replicability, something which has never been achieved by earlier methods. Accordingly, described herein are methods and compositions relating to such assays. The inventors have further identified cut-off values and risk thresholds for these assays, which permit early and surprisingly accurate detection of transplant rejection in a way that dramatically reduces the effects of operator or site variability. That is, in sharp contrast to earlier methods, the inventors have provided methods and compositions relating to transplant rejection which are universal and not specific to a single location, study group, or assay operator.

In some aspects of any of the embodiments, described herein are methods of identifying a kidney transplant patient as being stable and not in transplant rejection, methods of treating kidney transplant rejection in a patient who has received a kidney transplant, and methods of detecting kidney tissue (e.g., kidney tissue from a kidney transplant), that is undergoing pathological apoptosis.

### Transplantation

As used herein, a “transplant” is when cells, tissue, and/or an organ or organs are implanted in another part of the subject or in a different subject. This can also include, but is not limited to an engraftment. Transplantation is performed by a method or route which results in at least partial localization of the introduced cells, tissue, and/or organ(s) at a desired site, such as a site of injury or repair, such that a desired effect(s) is produced. The period of viability of the cells, tissue, or organ after administration to a subject can be as short as a few hours, e.g., twenty-four hours, to a few days, to as long as several years, (i.e., long-term engraftment or transplant). In some embodiments, the cells, tissue, and/or organ(s) that are being transplanted are kidney cells, tissue, and/or organ(s). The kidney cells, tissue, and/or organ can be obtained from a donor mammal, preferably from a donor human. The kidney cells, tissue, and/or organ can be obtained from stem cells or 3D culture.

Transplantation occurs when a subject has a need of kidney cells, tissue, and/or organ(s). The need of kidney cells, tissue, and/or organ(s) can result from acute or chronic injury. The acute or chronic injury can come from a kidney disease, disorder, or condition. Examples of kidney disease, disorder, or condition include, but are not limited to renal fibrosis, Alport syndrome, autosomal dominant polycystic kidney disease, chronic kidney disease, IgA nephropathy, type 1 diabetes, type 2 diabetes mellitus, focal segmental glomerulosclerosis, and nephropathy.

As used herein, “transplant rejection” is when the transplanted cells, tissues, and/or organ(s) are rejected by the subject's immune system, which then destroys the transplanted tissue. In some embodiments, the transplant rejection is a kidney transplant rejection. There are three kinds of transplant rejection: hyperacute rejection, acute rejection, and chronic rejection. Hyperacute rejection is a form of rejection that manifests itself in the minutes to hours following transplantation. Acute rejection occurs within weeks to months after transplantation, with most rejections occurring within the first three months to one year after transplantation. Chronic rejection leads to tissue destruction over the course of months or years after transplantation. Symptoms of a kidney transplant rejection include, but are not limited to a body temperature above 100° F. (37.8° C.), pain or tenderness around the transplant, swelling of the hands, feet, or eyelids, rapid weight gain, reduced urine output, and flu-like symptoms. Diagnosis of a transplant rejection include laboratory data such as blood or tissue biopsy examining infiltrating T cells, eosinophils, plasma cells, and neutrophils, structural compromise of tissue anatomy and injury to blood vessels. Diagnosis may also include cellular magnetic resonance imaging (MRI) of immune cells radiolabeled in vivo and/or gene expression profiling (GEP). In some embodiments, diagnosis of a transplant rejection can include PROTEINSIMPLE'S ELLA technology as described herein.

Treatment of a transplant rejection (e.g., rejection therapy) includes, but may not be limited to a test to identify disease in the graft, a renal biopsy, administration of methylprednisolone, administration of plasma exchange, administration of intravenous immunoglobulin, administration of anti-thymocyte globulin, administration of an anti-CD20 antibody, administration of an lymphocyte-depleting antibody, administration of one or more immunosuppressive drugs and/or therapies, administration of antibody drugs, administration of a blood transfer, administration of a marrow transplant, and administration of gene therapy. These treatments may be used as stand alone or in combination with each other. In some embodiments of any of the aspects, treatment of a transplant rejection (e.g., rejection therapy) includes, but may not be limited to administration of methylprednisolone, administration of plasma exchange, administration of intravenous immunoglobulin, administration of anti-thymocyte globulin, administration of an anti-CD20 antibody, and administration of a lymphocyte-depleting antibody. These treatments may be used as stand alone or in combination with each other. Rejection treatments are well known to those in the art. Those skilled in the art will be able to select and use the best rejection treatment for the subject in need thereof.

As used herein, a “graft” is the transplantation or implantation of cells and/or tissues surgically into an organ or body part to replace a damaged part or compensate for a defect. Identifying disease in the graft can comprise identifying either an acute disease or a chronic disease. One who is skilled in the art will be familiar with grafting and identifying disease in a graft.

As used herein, a renal biopsy is a medical procedure in which a small piece of kidney is removed from the body for examination, usually under a microscope. Microscopic examination of the tissue can provide information needed to diagnose, monitor, or treat problems of the kidney. A renal biopsy can be targeted to a particular area, such as a lesion. A renal biopsy can be non-targeted. If the renal biopsy is non-targeted, any sufficiently sized piece of kidney tissues that is the equivalent of 2-25 μL can be used. The renal biopsy can be a native renal biopsy where the patient's own kidneys are biopsied. The renal biopsy can be a transplant renal biopsy, where the transplanted kidney in the patient is biopsied.

Transplanted kidney biopsies can be performed when nothing is apparently wrong with the transplanted kidney which is also known as a protocol transplant biopsy. A protocol transplant biopsy can occur at 0 months, at 1 month, at 2 months, at 3 months, at 4 months, at 5 months, at 6 months, at 7 months, at 8 months, at 9 months, at 10 months, at 11 months, at 12 months or longer post-transplant. A biopsy of the transplanted kidney taken during the transplant operation is also known as an implantation transplant biopsy or post-perfusion transplant biopsy. If the transplanted kidney is not working properly, a biopsy of the malfunctioning kidney is also known as an indication transplant biopsy. A renal biopsy can be performed with the aid of “real-time” medical imaging to guide the positioning of biopsy equipment which is also known as imaging-guided renal biopsy. Alternatively, a renal biopsy can be performed without imaging-guidance using indirect assessments of position such as “needle-swing” to confirm appropriate placement of biopsy equipment and is also known as blind renal biopsy.

Methylprednisolone is a corticosteroid that works on the immune system to relieve swelling, redness, itching, and allergic reactions. One who is skilled in the art will know how to administer methylprednisolone, e.g., intravenously to provide rejection therapy.

As used herein, “plasma exchange” refers to a procedure that separates plasma from blood cells using a machine in order to remove autoantibodies, immune complexes, cytokines, and other inflammatory mediators. The blood cells are then mixed with a plasma substitute and placed back into the subject to prevent, treat, or reduce rejection of the transplanted organ. One who is skilled in the art will be familiar with plasma exchange and how to administer such therapies to a subject to prevent, treat, or reduce rejection of cells, tissues, and/or organs.

Intravenous immunoglobulin is made from a pool of immunoglobulins from the plasma of healthy donors and is administered to prevent, treat, or reduce the rejection of the transplanted organ and to reduce inflammation. One who is skilled in the art will be familiar with the use of intravenous immunoglobulin and how to administer is to a subject to prevent, treat, or reduce rejection of cells, tissues, and/or organs.

Anti-thymocyte globulin is an infusion of antibodies (e.g., horse or rabbit-derived) against human T cells and their precursors (thymocytes), which is used in the prevention and treatment of acute rejection in organ transplantation. In some embodiments of any of the aspects, anti-thymocyte globulin comprises polyclonal antibodies. One who is skilled in the art will be familiar with anti-thymocyte globulin and how to administer to a subject to prevent, treat, or reduce rejection of cells, tissues, and/or organs.

Anti-CD20 antibody and/or anti-CD20 therapy is the use of monoclonal antibodies specific to CD20, which are administered to deplete of circulating CD20-positive B cells in the periphery. Exemplary anti-CD20 antibodies include but are not limited to rituximab, ocrelizumab, ofatumumab, and obinutuzumab. One who is skilled in the art will be familiar with anti-CD20 antibodies and how to administer them to a subject to prevent rejection of cells, tissues, and/or organs.

Lymphocyte-depleting antibodies are monoclonal antibodies that target lymphocytes, which mediate transplant rejection and reduce immunogenic tolerance. Exemplary lymphocyte-depleting antibodies include but are not limited to muromonab. One who is skilled in the art will be familiar with lymphocyte-depleting antibodies and how to administer them to a subject to prevent, treat, or reduce rejection of cells, tissues, and/or organs.

As used herein “immunosuppression therapy” refers to a therapeutic treatment used to reduce the activation or efficacy of the immune system. Immunosuppression therapy can reduce or completely shut down the activation of the immune system. Exemplary immunosuppression therapies include but are not limited to belatacept, everolimus, prednisone, tacrolimus, mycophenolate, prednisolone, cyclosporine, siroliums, azathioprine corticosteroids, calcineurin inhibitors, mTOR inhibitors, or a combination thereof. Exemplary examples of immunosuppressive drugs include, but are not limited to corticosteroids (e.g., Prednisolone, Hydrocortisone), calcineurin inhibitors (e.g., Ciclosporin, Tacrolimus), anti-proliferatives (e.g., Azathioprine, Mycophenolic acid), and mTOR inhibitors (e.g, Sirolimus, Everolimus). In some embodiments of any of the aspects, immunosuppression therapy comprises corticosteroids, calcineurin inhibitors, mTOR inhibitors, or a combination thereof. In some embodiments of any of the aspects, immunosuppression therapy comprises one or more of belatacept, everolimus, prednisone, tacrolimus, mycophenolate, prednisolone, cyclosporine, siroliums, and azathioprine. One who is skilled in the art will be familiar with how to administer such therapies to a patient as to prevent, treat, or reduce transplant rejection of the cells, tissues, and/or organ(s) as described herein.

As used herein, “antibody drugs” refers to monoclonal antibodies that are engineered to restore, enhance, suppress, modify, or mimic the immune system. In some embodiments of any of the aspects, antibody drugs are used in the methods described herein to suppress the immune response. Exemplary examples of antibody drug treatments include, but are not limited to monoclonal anti-IL-2Rα receptor antibodies (e.g., Basiliximab, Daclizumab), monoclonal anti-IL-6R receptor antibodies (e.g., Tocilizumab), polyclonal anti-T-cell antibodies (e.g., Anti-thymocyte globulin (ATG), Anti-lymphocyte globulin (ALG)), and monoclonal anti-CD20 antibodies (rituximab, ocrelizumab, ofatumumab, or obinutuzumab). One who is skilled in the art will be familiar with antibody drugs and how to administer them to a subject to prevent, treat, or reduce rejection of cells, tissues, and/or organs.

Blood transfer or blood transfusion is a routine medical procedure in which donated blood is provided to a patient through a narrow tube placed within a vein, e.g., a vein in an arm. Blood transfers can be blood transfusions that are sometimes treated with photopheresis or extracorporeal photoimmune therapy (ECP) to remove antibody molecules specific to the transplanted tissue. One who is skilled in the art will be familiar with blood transfer and how to administer them to a subject to prevent, treat, or reduce rejection of cells, tissues, and/or organs.

As used herein, “marrow transplant” refers to a procedure that infuses healthy blood-forming stem cells into a patient's body to replace bone marrow that is not producing enough healthy blood cells or which is producing harmful or deleterious blood cells. Marrow transplants or bone marrow transplants can replace the transplant recipient's immune system with the donor's, and the recipient will then accept the new organ without rejection. In some embodiments, the marrow's hematopoietic stem cells must be from the individual who donated the transplant or from an identical twin or a clone of the donor. One who is skilled in the art would be able to perform this procedure to prevent, treat, or reduce transplant rejection of the cells, tissues, and/or organ(s) as described herein.

As used herein, “gene therapy” refers to methods that target the genes that cause the body to reject transplants, e.g., by deactivating such genes. Examples of genes targeted for gene therapy include huntingtin (HTT) gene and breast cancer type 1 (BRCA1) gene. One who is skilled in the art will be familiar with gene therapy and how to administer such therapies to a subject to prevent, treat, or reduce rejection of cells, tissues, and/or organs.

In some embodiments of any of the aspects, the rejection therapy can comprise a test to identify disease in the graft, a renal biopsy, methylprednisolone, plasma exchange, intravenous immunoglobulin, anti-thymocyte globulin, anti-CD20 antibody, lymphocyte-depleting antibody, or a combination thereof. In some embodiments of any of the aspects, the rejection therapy can comprise administering methylprednisolone, plasma exchange, intravenous immunoglobulin, anti-thymocyte globulin, anti-CD20 antibody, lymphocyte-depleting antibody, or a combination thereof to the patient.

In some embodiments of any of the aspects, a sample is used in the methods and assays described herein. In some embodiments, the sample is a urine sample. In some embodiments, the sample is a kidney sample. A kidney sample can include, but not be limited to, kidney cells, kidney tissue, blood from the kidney, urine, and a kidney organ.

As used herein, “sample” refers to a sample with an unknown concentration of one or more analytes (e.g. proteins). As used herein, “standard” or “standard sample” refers to a sample with a known concentration of one or more analytes, from which a standard curve can be built. As used herein, “control” or “control sample” refers to a sample with a known concentration of protein. As used herein, “spike” or “spike sample” refers to a biological sample with a known amount of protein added.

The sample collected can be a cell sample, a tissue sample, an organ sample, or a urine sample. In some embodiments, the sample collected is a urine sample. The urine sample can be fresh, stored over time at room temperature, or stored over time in a refrigerator or freezer. The urine sample can also comprise kidney cells and/or kidney tissue. The urine sample can be collected through a catheter, through excretion into a collection tube, or through surgical removal of kidney tissue and/or organ. The urine sample can comprise of additional proteins. The urine sample can be collected in the morning (also known as a first morning specimen and/or an eight-hour specimen). The urine sample can be collected during the day. The urine sample can be collected at night. The urine sample can be collected at any time (also known as a random specimen). The urine sample can be a timed-collection sample where samples are collected at specific times over at least a 1 hour period, at least a 2 hour period, at least a 3 hour period, at least a 4 hour period, at least a 5 hour period, at least a 6 hour period, at least a 7 hour period, at least an 8 hour period, at least a 9 hour period, at least a 10 hour period, at least an 11 hour period, at least a 12 hour period, at least a 13 hour period, at least a 14 hour period, at least a 15 hour period, at least a 16 hour period, at least a 17 hour period, at least a 18 hour period, at least a 19 hour period, at least a 20 hour period, at least a 21 hour period, at least a 22 hour period, at least a 23 hour period, at least a 24 hour period or more. The urine sample can be a 24-hours urine sample. A 24-hours urine sample is a lab test that measure what is in the urine. The urine is collected in a special container over a full 24-hour period. The container is kept cool until the urine is returned to the lab. The urine sample can be a first part urine sample. A first part urine sample is the stream of urine that first leaves the urethra. The urine sample can be a mid-stream sample. The urine sample can be a last part urine sample. The urine sample can also be used for the following laboratory tests including, but not limited to analyte, albumin, aldosterone, quantitative amino acids, Aminolaevulinic acid (ALA), amylase, arsenic, β2-microglobulin, benzene metabolite, C-Peptide, urine, cadmium, calcium, cannabinoids: creatinine ratio, catecholamines, (fractionated, total), chloride, chromium, citric acid, cobalt, copper, free cortisol, creatine, creatinine, cyclic AMP, cysteine, glucose, heavy metals, histamine, homovanillic acid (HVA), hydroxyindoleacetic acid (HIAA), immunofixation, lead magnesium, mercury, metanephrines (total and fractionated), myoglobin, nickel, osmolality, oxalate, phenol, phosphorus, porphobilinogen (PBG), porphyrins, potassium, pregnancy test, total protein, selenium, urea nitrogen, uric acid, vanillylmandelic acid (VMA), xylose tolerance, and zinc. The urine sample can also be subjected to a urinalysis. A urinalysis is a test that examines urine by physical, chemical, or microscopic methods. A urinalysis can be used to diagnose conditions including, but not limited to bladder stones, kidney stones, kidney failure, urinary tract infection, nephritis, epididymitis, injury to the kidney, bladder, urethra, or ureter, irritable bladder, kidney disease, orchitis, prostate cancer, prostatitis, pyelonephritis, proteinuria, retrograde ejaculation, urinary incontinence, ureterocele, urethral stricture, urethral stricture, urethritis, and Wilms tumor. A physical test of the urine will examine the color and clarity of the urine sample. A normal sample is usually clear and a light yellow color. A chemical test will measure the pH of the sample as well as the presence of proteins, sugars, nitrites, bilirubin, and other substances. A microscopic analysis examines the presence of crystals, bacterial, and other compounds (e.g., white blood cells) in the sample. Exemplary urinary proteins that can be found in a urine sample include, but are not limited to creatinine, albumin, cytokines, and bilirubin. In some embodiments, the urinary protein levels are normalized to urinary creatinine protein levels.

Some aspects of the technology described herein relate to detecting pathological apoptosis and/or detecting kidney tissue that is undergoing pathological apoptosis. Pathological apoptosis refers to a process of cell death in which the cell death impairs the normal function of the affected tissue. Pathological apoptosis can occur where cells are exposed to various stressors, including energy failure, oxidative stress, and unbalanced ion fluxes, resulting in parallel triggering and potential overshooting over several different cell death pathways. The different pathways interact with one another and result in complex patterns of biochemical manifestations and cellular morphological features. Features of programmed cell death such as pyknosis, nuclear condensation, and caspase-3 activation are combined with non-programmed cell death features.

Stable refers to a cell(s), a tissue(s), an organ(s), or a patient that is not undergoing transplant rejection. In some embodiments of any of the aspects, a stable transplant or stable patient is not experiencing pathological apoptosis. In some embodiments of any of the aspects, a stable transplant or stable patient is not in need of treatment for transplant rejection.

In some aspects of any of the embodiments, described herein are methods for identifying a kidney transplant patient as being stable and not in transplant rejection. In some embodiments of any of the aspects, the method can comprise:
a. contacting a urine sample with antibodies specific for each of a CXCL9, CXCL10, CCL2, and VEGF-A protein;
b. detecting the amount of each of CXCL9, CXCL10, CCL2, and VEGF-A bound to the antibodies, providing a data set comprising values for the level of each of CXCL9, CXCL10, CCL2, and VEGF-A detected;
c. analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant;
   wherein the weighting parameters are, 0.73797 for CXCL9, −0.046218 for CXCL10, 0.020161 for CCL2, and −0.21309 for VEGF-A and the constant is −4.0624; thereby providing a score; and
d. identifying a kidney transplant patient as being stable and not in transplant rejection based on the score being below a predetermined threshold.

In some embodiments, the method comprises:
a. contacting a urine sample with antibodies specific for each of CXCL9, CXCL10, CCL2, and VEGF-A proteins;
b. detecting the amount of each of CXCL9, CXCL10, CCL2, and VEGF-A bound to the antibodies, providing a data set comprising values for the level of each of CXCL9, CXCL10, CCL2, and VEGF-A detected;
c. analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant; and
   the data set further comprises a value for the time from transplant of the kidney and the weighting process further comprises applying a weighting parameter for the time from transplant of the kidney,
   wherein the weighting parameters are 0.75542 for CXCL9, 0.057341 for CXCL10, 0.095251 for CCL2, −0.34348 for VEGF-A, and 0.71722 for time from transplant; and
   wherein the constant is −7.1573 thereby providing a score; and
d. identifying a kidney transplant patient as being stable and not in transplant rejection based on the score being below a predetermined threshold.

In some embodiments of any of the aspects, the method comprises:
a. contacting a urine sample with antibodies specific for each of a CXCL9, CXCL10, CCL2, and VEGF-A protein;
b. detecting the amount of each of CXCL9, CXCL10, CCL2, and VEGF-A bound to the antibodies, providing a data set comprising values for the level of each of CXCL9, CXCL10, CCL2, and VEGF-A detected; and
c. analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant;
   wherein the weighting parameters are, 0.73797 for CXCL9, −0.046218 for CXCL10, 0.020161 for CCL2, and −0.21309 for VEGF-A and the constant is −4.0624; thereby providing a score.

In some embodiments, the method comprises:
a) contacting a urine sample with antibodies specific for each of CXCL9, CXCL10, CCL2, and VEGF-A proteins;
b) detecting the amount of each of CXCL9, CXCL10, CCL2, and VEGF-A bound to the antibodies, providing a data set comprising values for the level of each of CXCL9, CXCL10, CCL2, and VEGF-A detected; and
c) analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant; and
   the data set further comprises a value for the time from transplant of the kidney and the weighting process further comprises applying a weighting parameter for the time from transplant of the kidney,
   wherein the weighting parameters are 0.75542 for CXCL9, 0.057341 for CXCL10, 0.095251 for CCL2, −0.34348 for VEGF-A, and 0.71722 for time from transplant; and
   wherein the constant is −7.1573, thereby providing a score.

In some embodiments of any of the aspects, the predetermined thresholds are the 75% percentile Score 4 scores for normal patients in Table 1, e.g., −1.3514 for a patient of any age, or −1.9191 for patients under 18 years of age, or −1.1116 for a patient greater than 18 years of age. In some embodiments of any of the aspects, the predetermined thresholds are the 75% percentile Score4 scores for borderline patients in Table 1, e.g., −1.1599 for a patient of any age, or −1.1887 for patients under 18 years of age, or −1.1547 for a patient greater than 18 years of age.

In some aspects of any of the embodiments, described herein are methods of treating kidney transplant rejection in a patient who has received a kidney transplant and/or detecting kidney tissue undergoing pathological apoptosis. In some embodiments of any of the aspects, the methods comprise:
a) administering rejection therapy to a patient determined to have:
   i) a urinary protein level of CXCL9 greater than 78.832 pg/mL;
   ii) a urinary protein level of CXCL10 greater than 8.5748 pg/mL;
   iii) a urinary protein level of CCL2 greater than 205.67 pg/mL; and
   iv) a urinary protein level of VEF-A greater than 142.98 pg/mL; and
b) administering standard immunosuppression therapy to a patient determined not to have:
   i) a urinary protein level of CXCL9 greater than 78.832 pg/mL;
   ii) a urinary protein level of CXCL10 greater than 8.5748 pg/mL;
   iii) a urinary protein level of CCL2 greater than 205.67 pg/mL; and
   iv) a urinary protein level of VEF-A greater than 142.98 pg/mL.

In some embodiments of any of the aspects, the methods comprise:
a) administering rejection therapy to a patient determined to have:
   i) a urinary protein level of CXCL9 greater than a normal or borderline level in Table 1;
   ii) a urinary protein level of CXCL10 greater than a normal or borderline level in Table 1;
   iii) a urinary protein level of CCL2 greater than a normal or borderline level in Table 1; and
   iv) a urinary protein level of VEF-A greater than a normal or borderline level in Table 1; and
b) administering standard immunosuppression therapy to a patient determined not to have:
   i) a urinary protein level of CXCL9 greater than a normal or borderline level in Table 1;
   ii) a urinary protein level of CXCL10 greater than a normal or borderline level in Table 1;
   iii) a urinary protein level of CCL2 greater than a normal or borderline level in Table 1; and
   iv) a urinary protein level of VEF-A greater than a normal or borderline level in Table 1.

In some embodiments of any of the aspects, the normal or borderline level in Table 1 is the 75% tile normal or borderline level. In some embodiments of any of the aspects, the methods comprise:
a) contacting a urine sample obtained from the kidney tissue with antibodies specific for each of CXCL9, CXCL10, CCL2, and VEGF-A proteins;
b) detecting the amount of each of CXCL9, CXCL10, CCL2, and VEGF-A bound to the antibodies, providing a data set comprising values for the level of each of CXCL9, CXCL10, CCL2, and VEGF-A detected.

The kidney tissue can be identified as undergoing apoptosis, or the subject as being in need of kidney transplant rejection treatment if the score is above a predetermined threshold. In some embodiments of any of the aspects, the detecting further comprises analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, optionally wherein the weighting parameters are, 0.73797 for CXCL9, −0.046218 for CXCL10, 0.020161 for CCL2, and −0.21309 for VEGF-A, and optionally wherein the constant is −4.0624. In some embodiments of any of the aspects, the data set further comprises a value for the time from transplant of the kidney, optionally wherein the weighting process further comprises applying a weighting parameter for the time from transplant of the kidney, optionally wherein the weighting parameters are 0.75542 for CXCL9, 0.057341 for CXCL10, 0.095251 for CCL2, −0.34348 for VEGF-A, and 0.71722 for time from transplant. In some embodiments of any of the aspects, the weighting process further comprises applying a constant, optionally wherein the constant is −7.1573.

In one aspect of any of the embodiments, described herein is a method of treating kidney transplant rejection in a patient who has received a kidney transplant, the method comprising: determining a urinary protein level of CXCL9; a urinary protein level of CXCL10; a urinary protein level of CCL2; and a urinary protein level of VEGF-A of a urine sample obtained from the kidney transplant; predicting whether the patient will experience an acute rejection based on an acute rejection prediction score output from a logistic regression model, wherein the urinary protein level of CXCL9; the urinary protein level of CXCL10; the urinary protein level of CCL2; and the urinary protein level of VEF-A measured from the patient are input into the logistic regression model; administering rejection therapy to the patient if the acute rejection prediction score exceeds a predetermined threshold; and administering standard immunosuppression therapy to the patient if the acute rejection prediction score does not exceed a predetermined threshold. In one aspect of any of the embodiments, described herein is a method of treating kidney transplant rejection in a patient who has received a kidney transplant, the method comprising: determining a urinary protein level of CXCL9; a urinary protein level of CXCL10; a urinary protein level of CCL2; and a urinary protein level of VEGF-A of a urine sample obtained from the kidney transplant; calculating an acute rejection prediction score output from a logistic regression model, wherein the urinary protein level of CXCL9; the urinary protein level of CXCL10; the urinary protein level of CCL2; and the urinary protein level of VEF-A measured from the patient are input into the logistic regression model; administering rejection therapy to the patient if the acute rejection prediction score exceeds a predetermined threshold; and administering standard immunosuppression therapy to the patient if the acute rejection prediction score does not exceed a predetermined threshold.

As detailed above, methods described herein relate to detecting the level of CXCL9, CXCL10, CCL2, and VEGF-A. In some embodiments of any of the aspects, the level of each of CXCL9, CXCL10, CCL2, and VEGF-A is detected or measured.

C—X—C motif chemokine ligand 9 (CXCL9) is a cytokine that stimulates immune cells through Th1 polarization and activation. Th1 cells produce IFN-γ, TNF-α, IL-2 and stimulate CTLs, NK cells, and macrophages. Sequences are known for CXCL9 genes and polypeptides for a number of species, e.g., human CXCL9 (NCBI Gene ID No: 4283) mRNA (e.g., NCBI Ref Seq: NM_002416.3) and polypeptide (e.g., NCBI Ref Seq: NP_002407.1). In some embodiments of any of the aspects, a CXCL9 protein is a protein with at least 80%, at least 85%, at least 90%, at least 95%, at least 98%, or 100% sequence identity to a CXCL9 polypeptide sequence provided for NCBI Gene ID No: 4283 as of Jan. 9, 2023.

C—X—C motif chemokine ligand 10 (CXCL10) is a cytokine that is secreted by several cell types in response to IFNγ including, but not limited to monocytes, endothelial cells, and fibroblasts. It can have a role in chemoattraction for monocytes, macrophages, T cells, NK cells, and dendritic cells, promotion of T cell adhesion to endothelial cells, antitumor activity, and inhibition of bone marrow colony formation and angiogenesis. Sequences are known for CXCL10 genes and polypeptides for a number of species, e.g., human CXCL10 (NCBI Gene ID No: 3627) mRNA (e.g., NCBI Ref Seq: NM_001565.4) and polypeptide (e.g., NCBI Ref Seq: NP_001556.2). In some embodiments of any of the aspects, a CXCL10 protein is a protein with at least 80%, at least 85%, at least 90%, at least 95%, at least 98%, or 100% sequence identity to a CXCL10 polypeptide sequence provided for NCBI Gene ID No: 3627 as of Jan. 9, 2023.

C—C motif chemokine ligand 2 (CCL2) is recruited by monocytes, memory T cells, and dendritic cells to the site of inflammation produced by either tissue injury or infection. Sequences are known for CCL2 genes and polypeptides for a number of species, e.g., human CCL2 (NCBI Gene ID No: 6347) mRNA (e.g., NCBI Ref Seq: NM_002982.4) and polypeptide (e.g., NCBI Ref Seq: NP_002973.1). In some embodiments of any of the aspects, a CCL2 protein is a protein with at least 80%, at least 85%, at least 90%, at least 95%, at least 98%, or 100% sequence identity to a CCL2 polypeptide sequence provided for NCBI Gene ID No: 6347 as of Jan. 9, 2023.

Vascular endothelial growth factor A (VEGF-A) is a dimeric glycoprotein that plays a significant role in neurons and is considered to be the main, dominant inducer to the growth of blood vessels. Sequences are known for VEGF-A genes and polypeptides for a number of species, e.g., human VEGF-A (NCBI Gene ID No: 7422) mRNA (e.g., NCBI Ref Seq: NM_001025366.3, NM_001025367.3, NM_001025368.3, NM_001025369.3, NM_001025370.3, NM_001033756.3, NM_001171622.2, NM_001171623.2, NM_001171624.2, NM_001171625.2, NM_001171626.2, NM_001171627.2, NM_001171628.2, NM_001171629.2, NM_001171630.2, NM_001204384.2, NM_001204385.2, NM_001287044.2, NM_001317010.1, NM_003376.6) and polypeptide (e.g., NCBI Ref Seq: NP_001020537.2, NP_001020538.2, NP_001020539.2, NP_001020540.2, NP_001020541.2, NP_001028928.1, NP_001165093.1, NP_001165094.1, NP_001165095.1, NP_001165096.1, NP_001165097.1, NP_001165098.1, NP_001165099.1, NP_001165100.1, NP_001165101.1, NP_001191313.1, NP_001191314.1, NP_001273973.1, NP_001303939.1, NP_003367.4). In some embodiments of any of the aspects, a VEGF-A protein is a protein with at least 80%, at least 85%, at least 90%, at least 95%, at least 98%, or 100% sequence identity to a VEGF-A polypeptide sequence provided for NCBI Gene ID No: 7422 as of Jan. 9, 2023.

In some embodiments of any of the aspects, the measurement and/or detection of CXCL9, CXCL10, CCL2, and VEGF-A is performed by contacting a sample (e.g., a urine sample) with at least one antibody reagent specific for each of CXCL9, CXCL10, CCL2, and VEGF-A.

In some embodiments of any of the aspects, measurement of the level of a target and/or detection of the level or presence of a target, e.g. of an expression product (polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, e.g. a polypeptide can comprise contacting a sample obtained from a subject with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

Methods to measure gene expression products are known to a skilled artisan. For example, a peptide can be detected in a sample by introducing into a sample a labeled anti-peptide antibody and other types of detection agent. For example, the antibody can be labeled with a detectable marker whose presence in the sample is detected by standard imaging techniques.

In some embodiments of any of the aspects, one or more of the reagents (e.g. an antibody reagent) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments of any of the aspects, a detectable label can be a radiolabel including, but not limited to ³H, ¹²⁵I, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, CA. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

Assays using such antibody detection agents are commonly referred to as immunoassays. Immunoassays include biochemical tests that measure the concentration of a substance in a biological sample, typically a fluid sample such as urine, blood or serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

In some embodiments, the antibody contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments of any of the aspects, the assay can be a Western blot analysis. In some embodiments of any of the aspects, the assay can be PROTEINSIMPLE's SIMPLE WESTERN. Additional methodology relating to PROTEINSIMPLE's SIMPLE WESTERN technology are known in the art, e.g., in U.S. Pat. Nos. 9,229,001; 9,216,412; 9,546,932; 9,651,568; 9,700,889; 9,500,645; 9,855,735; 10,022,696; 10,065,403; 10,252,263; 10,220,385; 10,228,367; 10,786,800; 10,076,752; 10,414,143; 10,746,733 and in U.S. patent application Ser. No. 16/570,127. These references are incorporated herein in their entireties.

Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments of any of the aspects, the immunoassay can be a quantitative or a semi-quantitative immunoassay. Further details of such immunoassays can be found, e.g., U.S. Pat. Nos. 4,943,522; 6,485,982; 6,187,598; 5,770,460; 5,622,871; 6,565,808, 4,444,880; 4305,924; and 4,135,884 U.S. patent applications Ser. No. 10/278,676; U.S. Ser. No. 09/579,673 and U.S. Ser. No. 10/717,082, which are incorporated herein by reference in their entirety

In one embodiment, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In some embodiments of any of the aspects, the level of one, more than one, or all of the proteins is determined using PROTEINSIMPLE's ELLA and/or SIMPLE PLEX technology/assay. In some embodiments of any of the aspects, the contacting step comprises contacting a microfluidic device with the sample. In some embodiments of any of the aspects, the microfluidic device comprises one or more microfluidic channels. In some embodiments of any of the aspects, the one or more microfluidic channels comprise a plurality of detection locations. A detection location can comprise a capture antibody specific for a target biomolecule (e.g., CXCL9, CXCL10, CCL2, or VEGF-A), conjugated to a wall or support in the microfluidic channel. In some embodiments of any of the aspects, each detection location comprises a capture antibody(ies) specific for only one target biomolecule. When the channel is contacted with the sample, the target biomolecules will be bound by the capture antibody and immobilized at the detection location comprising the capture antibody. In some embodiments of any of the aspects, the microfluidic device comprises at least one detection location specific for each of CXCL9, CXCL10, CCL2, and VEGF-A. In some embodiments of any of the aspects, the microfluidic device comprises at least two detection locations specific for each of CXCL9, CXCL10, CCL2, and VEGF-A. In some embodiments of any of the aspects, the microfluidic device comprises at least three detection locations specific for each of CXCL9, CXCL10, CCL2, and VEGF-A. In some embodiments of any of the aspects, the microfluidic device comprises one or more branches or parallel microfluidic channels. Each branch or parallel channel can comprise one or more detection locations specific for a target biomolecule (e.g., CXCL9, CXCL10, CCL2, and VEGF-A), such that each biomolecule is bound to detected in a unique branch or parallel channel that does not bind or detect the other biomolecules.

The contacting and detecting steps can further comprise contacting the sample with one or more detection antibody reagents, e.g., antibody reagents specific for one or more of the biomolecules and comprises an detection reagent (e.g., a fluorescent label). The detection antibody reagents can bind to the target biomolecule-capture antibody complex at the one or more detection locations, thereby providing a detectable signal whose intensity reflects the amount of the biomolecule present in the sample. In some embodiments of any of the aspects, the detection antibody reagent is specific for one of the target biomolecules, e.g., one of CXCL9, CXCL10, CCL2, and VEGF-A. In some embodiments of any of the aspects, the detection antibody reagent and capture antibody reagent bind different epitopes of a target biomolecule, e.g., one of CXCL9, CXCL10, CCL2, and VEGF-A. Methods of detecting and measuring fluorescence intensity are well known in the art.

The detection locations referred to herein are also referred in some embodiments as “glass nano reactors (GNRs), e.g., in Protein Simple's ELLA technology.

In some embodiments of any of the aspects, the contacting and detecting steps can comprise one or more washing steps, e.g., to remove unbound target biomolecules and/or detection antibody reagents.

The amount of bound target biomolecule, e.g., the amount of target biomolecule in contact with a capture and detection antibody reagent is measured and can be stored, transmitted, and/or displayed as a data set. In some embodiments of any of the aspects, the contacting and detecting steps can be performed by an automated system, e.g., that provides automated flow through the microfluidic channel(s), automated flow of the detection antibody(ies), automated wash steps, automated incubation periods, and/or automated detection of the label at the detection locations (e.g., detection of fluorescence). The automated system can take up to 80 minutes from the time of sample entry to determine the amount of contact between the sample and the antibody of interest and generate a data set. The automated system can have a dynamic range of 3-4 logs. The automated system can have a sensitivity of at least <1 pg/mL. The automated system can produce at least 48 results (e.g., data points), at least 72 results, at least 128 results, at least 256 results or more.

In some embodiments of any of the aspects, the automated system described herein can comprise a removable portion comprising the one or more microfluidic channels and detection locations described herein. This removable portion can also be referred to as a cartridge herein. The cartridge can be a microfluidic device. The cartridge has at least one fluid flow channel comprises at least one detection location that is provided with capture agent, the detection location being positioned for exposure to fluid flows within the device for conducting an assay. In some embodiments of any of the aspects, the cartridge comprises a flexible sheet covering the flow channel and the detection location, and forming, at a selected region of the flow channel away from the detection location, a flexible diaphragm of a fluidic valve operable by pneumatic pressure. The cartridge can be a single plex cartridge. The single plex cartridge can test at least 1 sample or more, at least 2 samples or more, at least 3 samples or more, at least 4 samples or more, at least 5 samples or more, at least 6 samples or more, at least 7 samples or more, at least 8 samples or more, at least 9 samples or more, at least 10 samples or more, at least 11 samples or more, at least 12 samples or more, at least 13 samples or more, at least 14 samples or more, at least 15 samples or more, at least 16 samples or more, at least 17 samples or more, at least 18 samples or more, at least 19 samples or more, at least 20 samples or more, at least 21 samples or more, at least 22 samples or more, at least 23 samples or more, at least 24 samples or more, at least 25 samples or more, at least 26 samples or more, at least 27 samples or more, at least 28 samples or more, at least 29 samples or more, at least 30 samples or more, at least 31 samples or more, at least 32 samples or more, at least 33 samples or more, at least 34 samples or more, at least 35 samples or more, at least 36 samples or more, at least 37 samples or more, at least 38 samples or more, at least 39 samples or more, at least 40 samples or more, at least 41 samples or more, at least 42 samples or more, at least 43 samples or more, at least 44 samples or more, at least 45 samples or more, at least 46 samples or more, at least 47 samples or more, at least 48 samples or more, at least 49 samples or more, at least 50 samples or more, at least 51 samples or more, at least 52 samples or more, at least 53 samples or more, at least 54 samples or more, at least 55 samples or more, at least 56 samples or more, at least 57 samples or more, at least 58 samples or more, at least 59 samples or more, at least 60 samples or more, at least 61 samples or more, at least 62 samples or more, at least 63 samples or more, at least 64 samples or more, at least 65 samples or more, at least 66 samples or more, at least 67 samples or more, at least 68 samples or more, at least 69 samples or more, at least 70 samples or more, at least 71 samples or more, at least 72 samples or more. A single plex cartridge can only detect one target.

In some embodiments, the cartridge can be a multianalyte cartridge, e.g., the cartridge can be a multiplex cartridge. A multianalyte or multiplex cartridge can detect two or more targets. A multianalyte cartridge can use at least 1 analyte, at least 2 analytes, at least 3 analytes, at least 4 analytes or more. A multiplex cartridge can perform at least 2 immunoassays, at least 3 immunoassays, at least 4 immunoassays, at least 5 immunoassays, at least 6 immunoassays, at least 7 immunoassays, at least 8 immunoassays or more. The cartridge can be a customizable cartridge. The multiplex, multianalyte, or customizable cartridge can test at least 1 sample or more, at least 2 samples or more, at least 3 samples or more, at least 4 samples or more, at least 5 samples or more, at least 6 samples or more, at least 7 samples or more, at least 8 samples or more, at least 9 samples or more, at least 10 samples or more, at least 11 samples or more, at least 12 samples or more, at least 13 samples or more, at least 14 samples or more, at least 15 samples or more, at least 16 samples or more, at least 17 samples or more, at least 18 samples or more, at least 19 samples or more, at least 20 samples or more, at least 21 samples or more, at least 22 samples or more, at least 23 samples or more, at least 24 samples or more, at least 25 samples or more, at least 26 samples or more, at least 27 samples or more, at least 28 samples or more, at least 29 samples or more, at least 30 samples or more, at least 31 samples or more, at least 32 samples or more, at least 33 samples or more, at least 34 samples or more, at least 35 samples or more, at least 36 samples or more, at least 37 samples or more, at least 38 samples or more, at least 39 samples or more, at least 40 samples or more, at least 41 samples or more, at least 42 samples or more, at least 43 samples or more, at least 44 samples or more, at least 45 samples or more, at least 46 samples or more, at least 47 samples or more, at least 48 samples or more, at least 49 samples or more, at least 50 samples or more, at least 51 samples or more, at least 52 samples or more, at least 53 samples or more, at least 54 samples or more, at least 55 samples or more, at least 56 samples or more, at least 57 samples or more, at least 58 samples or more, at least 59 samples or more, at least 60 samples or more, at least 61 samples or more, at least 62 samples or more, at least 63 samples or more, at least 64 samples or more, at least 65 samples or more, at least 66 samples or more, at least 67 samples or more, at least 68 samples or more, at least 69 samples or more, at least 70 samples or more, at least 71 samples or more, at least 72 samples or more. In some embodiments, the cartridge is a customizable cartridge. The cartridge can contain a calibration curve.

In some embodiments of any of the aspects, the technology/assay utilizes at least one cartridge that comprises antibodies used to measure protein levels. In some embodiments of any of the aspects, the technology/assay utilizes at least one cartridge that detection antibodies and capture antibodies specific for one or more target biomolecules. The cartridge can be preloaded with a predetermined, and optionally equal amount of the detection antibodies and/or capture antibodies specific for each target biomolecule, e.g., each of CXCL9, CXCL10, CCL2, and VEGF-A.

The cartridge can test samples in duplicate. The cartridge can test samples in triplicate. At least 2.5 μL of sample is loaded onto a cartridge, at least 5 μL of sample is loaded onto a cartridge, at least 7.5 μL of sample is loaded onto a cartridge, at least 10 μL of sample is loaded onto a cartridge, at least 12.5 μL of sample is loaded onto a cartridge, at least 15 μL of sample is loaded onto a cartridge, at least 17.5 μL of sample is loaded onto a cartridge, at least 20 μL of sample is loaded onto a cartridge, at least 22.5 μL of sample is loaded onto a cartridge, at least 25 μL or more of sample is loaded onto a cartridge.

The detection antibody can be a fluorescent antibody. The cartridge can undergo at least one wash to remove unbound analyte and/or detection antibody. Additional methodology relating to cartridges are known in the art, e.g., in U.S. Pat. No. 9,216,412 and U.S. patent applications Ser. No. 16/570,127, 16/319,056, 17/674,623. These references are herein incorporated by reference in their entireties.

In some embodiments of any of the aspects, the detecting step comprises simultaneously detecting the amount of each of CXCL9, CXCL10, CCL2, and VEGF-A, e.g., the amount of CXCL9, CXCL10, CCL2, and VEGF-A bound to the capture and detection antibodies.

In some embodiments, the cartridge comprises a standard curve. The standard curve defines the values of the samples. In some embodiments, the standard curve is defined by a top concentration and dilution factor. The dilution factor can be 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, 1:100, 1:105, 1:110, 1:115, 1:120, 1:125, 1:130, 1:135, 1:140, 1:145, 1:150, 1:155, 1:160, 1:165, 1:170, 1:175, 1:180, 1:185, 1:190, 1:195, 1:200 or more. There is one standard curve for each analyte, which utilizes presently loaded Kit/Curve files from the PROTEINSIMPLE ELLA technology. In some embodiments, four data points can be selected to create the standard curve which is a 5-parameter sigmoid curve using a Levenberg-Marquardt algorithm.

In one aspect of any of the embodiments, described herein is a cartridge comprising one or more microfluidic channels, the channels comprising at least four distinguishable detection locations, each detection location comprising a capture antibody specific for CXCL9, CXCL10, CCL2, or VEGF-A and conjugated to a wall or support in the microfluidic channel. The at least four distinguishable detection locations collectively comprise capture antibodies specific for each of CXCL9, CXCL10, CCL2, or VEGF-A. The cartridge can further comprise detection antibodies specific for each of CXCL9, CXCL10, CCL2, or VEGF-A. The cartridge can further comprise buffers and wash reagents. In some embodiments, the cartridge is compatible with PROTEINSIMPLE ELLA.

The PROTEINSIMPLE ELLA technology can be combined with various technologies including, but not limited to PROTEINSIMPLE JESS, PROTEINSIMPLE ABBY, PROTEINSIMPLE WES, PROTEINSIMPLE PEGGY SUE, PROTEINSIMPLE SALLY SUE, PROTEINSIMPLE NANOPURE 1000, PROTEINSIMPLE SIMPLE PLEX ASSAYS, PROTEINSIMPLE MILO, PROTEINSIMPLE MFI 5000 SERIES, PROTEINSIMPLE FLUORCHEM M, PROTEINSIMPLE FLUORCHEM R, and PROTEINSIMPLE FLUORCHEM E.

Additional methodology related to PROTEINSIMPLE's ELLA technology are known in the art, e.g., in U.S. patent numbers: 9,229,001; 9,216,412; 9,546,932; 9,651,568; 9,700,889; 9,500,645; 9,855,735; 10,022,696; 10,065,403; 10,252,263; 10,220,385; 10,228,367; 10,786,800; 10,076,752; 10,414,143; 10,746,733 9,216,412 and U.S. patent applications Ser. No. 16/570,127, 16/319,056, 17/674,623. These references are herein incorporated by reference in their entirety.

Antibody refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

Antibody reagent refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

Antibodies and/or antibody reagents can include an immunoglobulin molecule, a monoclonal antibody, a chimeric antibody, a CDR-grafted antibody, a humanized antibody, a fully human antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof. Where antibodies are referred to herein, it is understood that an antibody reagent may be used.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Antibodies for the various targets described herein are commercially available and can be used for the purposes of the invention to measure protein expression levels. Alternatively, since the amino acid sequences for the targets described herein are known and publically available at the NCBI website, one of skill in the art can raise their own antibodies against these polypeptides of interest for the purpose of the methods described herein.

Exemplary antibodies commercially available for CXCL9 include, but are not limited to CXCL9 Recombinant Rabbit Monoclonal Antibody (11H1L14) (Cat. No. 701117, Invitrogen, Waltham, MA); CXCL9 Monoclonal Antibody (6) (Cat. No. MA5-30320, Invitrogen, Waltham, MA); Anti-MIG Antibody (A-9) (Cat. No. sc-514138, Santa Cruz Biotechnologies, Santa Cruz, CA); Anti-CXCL9 monoclonal antibody (AB17703) (Cat. No. CABT-28819MH, Creative Diagnostics, New York, NY); Anti-CXCL9 monoclonal antibody (NN0330-8G22) (Cat. No. CABT-28820MH, Creative Diagnostics, New York, NY); Anti-CXCL9 Monoclonal antibody, PE (NJH-3G6.6 [PE]) (Cat. No. CABT-BL7426, Creative Diagnostics, New York, NY).

Exemplary antibodies commercially available for CXCL10 include, but are not limited to CXCL10 Recombinant Rabbit Monoclonal Antibody (10H11L3) (Cat. No. 701225, Invitrogen, Waltham, MA); CXCL10 Monoclonal Antibody (4D5) (Cat. No. MA1-80897, Invitrogen, Waltham, MA); Anti-IP-10 Antibody (1) (Cat. No. sc-101500, Santa Cruz Biotechnologies, Santa Cruz, CA); Purified anti-human CXCL10 (IP-10) Antibody (J034D6) (Cat. No. 519501, BioLegend, San Diego, CA); Monoclonal Mouse anti-Human CXCL10/IP-10 Antibody (clone 4D5) (Cat. No. LS-C43826-MSS10, Lifespan Bioscience, Seattle, WA).

Exemplary antibodies commercially available for CCL2 include, but are not limited to MCP-1 Monoclonal Antibody (2D8) (Cat. No. MA5-17040, Invitrogen, Waltham, MA); CCL2 (MCP-1) Monoclonal Antibody (2H5), eBioscience™ (Cat. No. 14-7096-81, Invitrogen, Waltham, MA); Mcp1 Monoclonal Antibody (1B9F7) (Cat. No. 66272-1-IG, Proteintech, Rosemont, IL); MCP-1 Monoclonal Antibody (OTI2H5), Biotin (Cat. No. TA700025, OriGene, Rockville, MD); CCL2 Monoclonal Antibody (2E2) (Cat. No. H00006347-M08A, Abnova, Taipei, Taiwan); Anti-MCP-1-4/eotaxin Antibody (B-2) (Cat. No. sc-377082, Santa Cruz Biotechnologies, Santa Cruz, CA).

Exemplary antibodies commercially available for VEGFA include, but are not limited to Anti-VEGFA antibody [VG-1] (Cat. No. ab1316, Abcam, Cambridge, UK); VEGF Monoclonal Antibody (JH121) (Cat. No. MA5-13182, Invitrogen, Waltham, MA); VEGFA Monoclonal Antibody (2E2H9) (Cat. No. 66828-1-IG, Proteintech, Rosemont, IL); VEGF Monoclonal Antibody (OTI1A6), TrueMAB™ (Cat. No. TA803263, OriGene, Rockville, MD); Monoclonal Mouse anti-Human VEGFA/VEGF Antibody (clone JH121) (Cat. No. LS-C88112-MSS11, Creative Diagnostics, New York, NY).

The data set referenced above herein can be analyzed, e.g., by a control system comprising one or more processors and configured to execute machine executable code executing a logistical regression model. In some embodiments of any of the aspects, the technology described herein relates to a control system executing machine executable code (and computer readable media for causing computer systems) for obtaining/receiving data from at least one sample obtained from at least one subject, the system comprising 1) a measuring module configured to receive the at least one sample and perform at least one analysis on the at least one sample to determine the level (e.g. expression level and/or activity level) of CXCL9, CXCL10, CCL2, and VEGF-A in the sample; 2) a storage device configured to store data output from the determination module; and 3) a data set for displaying a content based in part on the data output from the determination module, wherein the content comprises a signal indicative of the level (e.g. expression level and/or activity level) of CXCL9, CXCL10, CCL2, and VEGF-A.

In one aspect of any of the embodiments, described herein is a system comprising: (a) at least one memory containing at least one computer program adapted to control the operation of the computer system to implement a method that includes a measuring module configured to measure the level of CXCL9, CXCL10, CCL2, and VEGF-A in a test sample obtained from a subject; a storage module configured to store output data from the determination module; a comparison module adapted to compare the data stored on the storage module with a reference level, and to provide a retrieved content, and a data set for displaying whether the sample comprises a level of CXCL9, CXCL10, CCL2, and VEGF-A which is significantly decreases relative to the reference expression level and/or displaying the relative level of CXCL9, CXCL10, CCL2, and VEGF-A and (b) at least one processor for executing the computer program.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; a tablet; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip. The term a “computer system” may refer to a system having a computer, where the computer comprises a computer-readable medium embodying software to operate the computer. The term “software” is used interchangeably herein with “program” and refers to prescribed rules to operate a computer. Examples of software include: software; code segments; instructions; computer programs; and programmed logic.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J #, Visual Basic, C, C #, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

Embodiments of the technology can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The functional modules of certain embodiments of the technology include at minimum a measuring module, a storage module, a control system, and a data set. The functional modules can be executed on one, or multiple, computers, or by using one, or multiple, computer networks. The measuring module has computer executable instructions to provide e.g., levels of expression products etc in computer readable form.

The measuring module can comprise any system for detecting a signal elicited from an assay to determine the level of CXCL9, CXCL10, CCL2, and VEGF-A as described above herein, e.g., for performing the contacting and detecting steps. This signal results from the sample's CXCL9, CXCL10, CCL2, and VEGF-A contacting the detection reagent. Both the contacting step and the detecting step described herein can be performed in an automated device. In some embodiments, such systems can include an instrument, e.g., PROTEINSIMPLE ELLA™ (BioTechne) as described herein for quantitative measurement of polypeptides. In some embodiments, the measuring module can measure the intensity of a detectable signal from an assay indicating the level of CXCL9, CXCL10, CCL2, and VEGF-A polypeptide in the test sample. In some embodiments, the assay can be an immunoassay.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, biomolecule assayed and the level of said biomolecule. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In some embodiments of any of the systems described herein, the storage module stores the output data from the determination module. In additional embodiments, the storage module stores reference information such as levels of CXCL9, CXCL10, CCL2, and VEGF-A in healthy subjects and/or a population of healthy subjects; levels of CXCL9, CXCL10, CCL2, and VEGF-A in stable subjects and/or a population of stable subjects; levels of CXCL9, CXCL10, CCL2, and VEGF-A in unstable subjects and/or a population of unstable subjects.

The “control system” can use a variety of available software programs and formats for computing the level of CXCL9, CXCL10, CCL2, and VEGF-A. Such algorithms are well established in the art. A skilled artisan is readily able to determine the appropriate algorithms based on the size and quality of the sample and type of data. The data analysis tools and equations described herein can be implemented in the control system of the invention. In one embodiment, the control system further comprises a comparison module, which compares the level of CXCL9, CXCL10, CCL2, and VEGF-A in a sample obtained from a subject as described herein with the mean value of CXCL9, CXCL10, CCL2, and VEGF-A in a population of healthy subjects. By way of an example, when the value of CXCL9, CXCL10, CCL2, and VEGF-A in a sample obtained from a subject is measured, a comparison module can compare or match the output data with the mean value of CXCL9, CXCL10, CCL2, and VEGF-A in a population of healthy subjects. In certain embodiments, the mean value of CXCL9, CXCL10, CCL2, and VEGF-A in a population of healthy subjects can be pre-stored in the storage module. In various embodiments, the comparison module can be configured using existing commercially-available or freely-available software for comparison purpose, and may be optimized for particular data comparisons that are conducted.

The control system, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware--as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). In some embodiments users can directly access data (via Hypertext links for example) residing on Internet databases using a HTML interface provided by Web browsers and Web servers.

The control system provides a computer readable comparison result that can be processed in computer readable form by predefined criteria, or criteria defined by a user, to provide content based in part on the comparison result that may be stored and output as requested by a user using an output module, e.g., a data set or score.

In some embodiments, the content displayed on the output module can be the level of CXCL9, CXCL10, CCL2, and VEGF-A in the sample obtained from a subject. In some embodiments, the content displayed on the data set can be the score described herein. In some embodiments, the content displayed on the output module can be the relative level of CXCL9, CXCL10, CCL2, and VEGF-A in the sample obtained from a subject as compared to the mean level of CXCL9, CXCL10, CCL2, and VEGF-A in a population of healthy, stable, or unstable subjects. In some embodiments, if the control system determines that the score is less or more than a threshold value described herein, the output module displays a signal indicating that the score is less or more than a threshold value described herein. In some embodiments, the signal indicates the subject is in need of treatment for transplant rejection. In some embodiments, the signal indicates the kidney tissue is undergoing apoptosis. In some embodiments, the signal indicates the the rejection category the patient is determined to belong to, e.g., stable, borderline, and/or acute rejection. In some embodiments, the signal indicates the degree to which the score exceeds or does not exceed the threshold value. In some embodiments, the content displayed on the output module can indicate whether the subject has an increased likelihood of having or developing transplant rejection. In some embodiments, the content displayed on the output module can be a numerical value indicating one of these risks or probabilities. In such embodiments, the probability can be expressed as a value. For example, rejection therapy can be administered to a patient determined to have a urinary protein level of CXCL9 greater than 78.832 pg/mL, a urinary protein level of CXCL10 greater than 8.5748 pg/mL, a urinary protein level of CCL2 greater than 205.67 pg/mL, a urinary protein level of VEF-A greater than 142.98 pg/mL and administering standard immunosuppression therapy to a patient determined not to have a urinary protein level of CXCL9 greater than 78.832 pg/mL, a urinary protein level of CXCL10 greater than 8.5748 pg/mL, a urinary protein level of CCL2 greater than 205.67 pg/mL, and a urinary protein level of VEF-A greater than 142.98 pg/mL. In some embodiments, the content displayed on the data set can be single word or phrases to qualitatively indicate a risk or probability. For example, a word “unlikely” can be used to indicate a lower risk for having or developing transplant rejection, while “likely” can be used to indicate a high risk for having or developing transplant rejection.

In one embodiment of any of the aspects, the content based on the control system result is displayed on a computer monitor. The control system result can also be a data set or score. In one embodiment of the invention, the content based on the data set or score is displayed through printable media. The output module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, California, or any other type of processor, visual display devices such as flat panel displays, cathode ray tubes and the like, as well as computer printers of various types.

In one embodiment, a World Wide Web browser is used for providing a user interface for display of the content based on the analyzing/comparing result. It should be understood that other modules of the invention can be adapted to have a web browser interface. Through the Web browser, a user can construct requests for retrieving data from the analyzing/comparing module. Thus, the user will typically point and click to user interface elements such as buttons, pull down menus, scroll bars and the like conventionally employed in graphical user interfaces.

Systems and computer readable media described herein are merely illustrative embodiments of the invention for determining the level of CXCL9, CXCL10, CCL2, and VEGF-A and/or a score as described herein in a sample obtained from a subject, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

As used herein, a “weighting parameter” is a value ranging between −1 and 1 and it identifies the proportional change between normal and acute rejection populations. In some embodiments of any of the aspects, the weighted parameter is between 0.70-0.80 for CXCL9, between −0.04 and −0.05 for CXCL10, between 0.02 and 0.03 for CCL2, and between −0.21 and −0.22 for VEGF-A. In some embodiments of any of the aspects, the weighted parameter is 0.73797 for CXCL9, −0.046218 for CXCL10, 0.020161 for CCL2, and −0.21309 for VEGF-A. In a logistical regression model, a weighting parameter can be applied to a level of a biomolecule by multiplying the level of the biomolecule by the weighting parameter.

As used herein, a “logistical regression model” is a statistical model that models the probability of one event (out of two alternatives) taking place by having the logarithm of the odds for the event be a linear combination of one or more independent variables and estimating the parameters of a logistic model. The logistical regression model applies one or both of parameters and a constant. The output of a logistical regression model can be a score. The parameters of a logistic regression can be estimated by maximum-likelihood estimation. In some embodiments, the parameters of a logistic regression are weighted parameters. In some embodiments, the constant can range between −1 to −10, between −2 to −9, between −3 to −8, between −3.5 to −7.5, between −2 to −10, between −3 to −10, between −3.5 to −10, between −1 to −7.5, between −2 to −7.5, between −3 to −7.5.

In some embodiments of any of the aspects, the logistic regression model includes a preliminary technical control step checking that the determined CXCL9 and CXCL10 levels are correlated to within a predetermined standard error (S.E.) value of a training set linear regression fit from one another with a predetermined slope and a predetermined y-intercept value. In some embodiments of any of the aspects, the urinary protein level of CXCL9; the urinary protein level of CXCL10; the urinary protein level of CCL2; the urinary protein level of VEF-A; and time from transplant are modified by adding a +1 pseudocount unit and by log 2-transforming the resulting values.

In some embodiments, the method can be performed immediately after the kidney transplant. In other embodiments, the method can be performed at least 1 minute after, at least 2 minutes after, at least 5 minutes after, at least 10 minutes after, at least 15 minutes after, at least 20 minutes after, at least 25 minutes after, at least 30 minutes after, at least 35 minutes after, at least 40 minutes after, at least 45 minutes after, at least 50 minutes after, at least 55 minutes after, at least one hour after, at least 2 hours after, at least 3 hours after, at least 4 hours after, at least 5 hours after, at least 6 hours after, at least 7 hours after, at least 8 hours after, at least 9 hours after, at least 10 hours after, at least 15 hours after, at least 20 hours after, at least 24 hours after, at least 25 hours after, at least 30 hours after, at least 35 hours after, at least 40 hours after, at least 45 hours after, at least 50 hours after, at least 55 hours after, at least 60 hours after, at least 65 hours after, at least 70 hours after, at least 75 hours after, at least 80 hours after, at least 85 hours after, at least 90 hours after, at least 95 hours after, at least 100 hours after, at least 105 hours after, at least 110 hours after, at least 115 hours after, at least 120 hours after, at least 2 days after, at least 3 days after, at least 4 days after, at least 5 days after, at least 6 days after, at least 7 days after, at least 8 days after, at least 9 days after, at least 10 days after, at least 11 days after, at least 12 days after, at least 13 days after, at least 14 days after, at least 15 days after, at least 16 days after, at least 17 days after, at least 18 days after, at least 19 days after, at least 20 days after, at least 21 days after, at least 22 days after, at least 23 days after, at least 24 days after, at least 25 days after, at least 26 days after, at least 27 days after, at least 28 days after, at least 29 days after, at least 30 days after, at least 31 days after, at least 32 days after, at least 33 days after, at least 34 days after, at least 35 days after, at least 36 days after, at least 37 days after, at least 38 days after, at least 39 days after, at least 40 days after, at least 41 days after, at least 42 days after, at least 43 days after, at least 44 days after, at least 45 days after, at least 46 days after, at least 47 days after, at least 48 days after, at least 49 days after, at least 50 days after, at least 51 days after, at least 52 days after, at least 53 days after, at least 54 days after, at least 55 days after, at least 56 days after, at least 57 days after, at least 58 days after, at least 59 days after, at least 60 days after, at least 61 days after, at least 2 months after, at least 3 months after, at least 4 months after, at least 5 months after, at least 6 months after, at least 7 months after, at least 8 months after, at least 9 months after, at least 10 months after, at least 11 months after, at least 12 months after, at least 1 year after, at least 2 years after, at least 3 years after, at least 4 years after, at least 5 years after, at least 6 years after, at least 7 years after, at least 8 years after, at least 9 years after, at least 10 years after or more.

In some embodiments of any of the aspects, the patient is a pediatric patient. In some embodiments of any of the aspects, the patient is less than 18 years old. In some embodiments of any of the aspects, the patient is less than 16 years old. In some embodiments of any of the aspects, the patient is less than 14 years old. In some embodiments of any of the aspects, the patient is less than 12 years old. In some embodiments of any of the aspects, the patient is less than 10 years old. In some embodiments of any of the aspects, the patient is less than 8 years old.

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of the target molecule in a population of subjects who do not have or are not diagnosed as having, and/or do not exhibit signs or symptoms of transplant rejection. In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing over time.

In some embodiments of any of the aspects, the level of expression products of no more than 200 other genes is determined. In some embodiments of any of the aspects, the level of expression products of no more than 100 other genes is determined. In some embodiments of any of the aspects, the level of expression products of no more than 20 other genes is determined. In some embodiments of any of the aspects, the level of expression products of no more than 10 other genes is determined.

In some embodiments of the foregoing aspects, the expression level of a given gene can be normalized relative to the expression level of one or more reference genes or reference proteins. In some embodiments of any of the aspects, the protein levels of the target biomolecules are normalized to urinary creatinine in the sample.

In some embodiments, the reference level can be the level in a sample of similar cell type, sample type, sample processing, and/or obtained from a subject of similar age, sex and other demographic parameters as the sample/subject for which the level of a marker is to be determined. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a urine sample from a subject. The term “test sample” also includes untreated or pretreated (or pre-processed) biological samples. The test sample can be obtained by removing a sample from a subject, but can also be accomplished by using a previously isolated sample (e.g. isolated at a prior timepoint and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject. In some embodiments of any of the aspects, the subject can be a subject in need of treatment for (e.g. having or diagnosed as having) transplant rejection or a subject at risk of or at increased risk of developing transplant rejection as described elsewhere herein.

In some embodiments of any of the aspects, the level of no more than 4 genes, markers, or proteins is determined. In some embodiments of any of the aspects, the level of no more than 5 genes, markers, or proteins is determined. In some embodiments of any of the aspects, the level of no more than 10 genes, markers, or proteins is determined. In some embodiments of any of the aspects, the level of no more than 20 genes, markers, or proteins is determined. In some embodiments of any of the aspects, the level of no more than 50 genes, markers, or proteins is determined. In some embodiments of any of the aspects, the level of no more than 100 genes, markers, or proteins is determined. In some embodiments of any of the aspects, the level of no more than 200 genes, markers, or proteins is determined.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of transplant rejection. A subject can be male or female.

A subject can be one who has been previously diagnosed with or identified as suffering from or having a condition in need of treatment (e.g. kidney transplant and/or kidney transplant rejection) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect of the full length polypeptide. Conservative substitution variants that maintain the activity of the wildtype will include a conservative substitution. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having acute kidney transplant rejection, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to acute rejection. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one substance. Exemplary delivery methods include, but are not limited to, direct delivery, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In all embodiments where a sample is obtained or has been obtained or provided, the sample can be sample taken, obtained, or provided via minimally invasive methods and/or involves only a minor intervention. In some embodiments of any of the aspects, a sample is taken, obtained, or provided by one or more of a blood draw or prick, an epidermal or mucus membrane swab, buccal sampling, saliva sample, an epidermal skin sampling technique, and/or collection of a secreted or expelled bodily fluid (e.g., mucus, urine, sweat, etc.), fecal sampling, semen/seminal fluid sampling, or clippings (e.g., of hair or nails). In some embodiments of any of the aspects, the sample comprises, consists of, or consists essentially of blood (or any fraction or component thereof), serum, urine, mucus, epithelial cells, saliva, buccal cells, a secreted or expelled bodily fluid, and/or hair or nail clippings.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The technology may be described in any one of the following numbered paragraphs:
Paragraph 1: A method of identifying a kidney transplant patient as being stable and not in transplant rejection, the method comprising: contacting a urine sample with antibodies specific for each of the CXCL9, CXCL10, CCL2, and VEGF-A proteins; detecting the amount of each of CXCL9, CXCL10, CCL2, and VEGF-A bound to the antibodies, providing a data set comprising values for the level of each of CXCL9, CXCL10, CCL2, and VEGF-A detected; analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant; wherein the weighting parameters are, 0.73797 for CXCL9, −0.046218 for CXCL10, 0.020161 for CCL2, and −0.21309 for VEGF-A and the constant is −4.0624; thereby providing a score; and identifying a kidney transplant patient as being stable and not in transplant rejection based on the score being below a predetermined threshold.
Paragraph 2: A method of identifying a kidney transplant patient as being stable and not in transplant rejection, the method comprising: contacting a urine sample with antibodies specific for each of CXCL9, CXCL10, CCL2, and VEGF-A proteins; detecting the amount of each of CXCL9, CXCL10, CCL2, and VEGF-A bound to the antibodies, providing a data set comprising values for the level of each of CXCL9, CXCL10, CCL2, and VEGF-A detected; analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant; and the data set further comprises a value for the time from transplant of the kidney and the weighting process further comprises applying a weighting parameter for the time from transplant of the kidney, wherein the weighting parameters are 0.75542 for CXCL9, 0.057341 for CXCL10, 0.095251 for CCL2, −0.34348 for VEGF-A, and 0.71722 for time from transplant; and wherein the constant is −7.1573 thereby providing a score; and identifying a kidney transplant patient as being stable and not in transplant rejection based on the score being below a predetermined threshold.
Paragraph 3: The method of any of the preceding paragraphs, wherein the contacting and detecting steps are performed in an automated device.
Paragraph 4: The method of paragraph 3, wherein the automated device comprises a cartridge comprising the antibodies.
Paragraph 5: The method of any of the preceding paragraph, wherein the detecting step comprises simultaneously detecting the amount of each of CXCL9, CXCL10, CCL2, and VEGF-A bound to the antibodies.
Paragraph 6: The method of any of the preceding paragraphs, wherein the urinary protein levels are normalized to urinary creatinine.
Paragraph 7: A method of treating kidney transplant rejection in a patient who has received a kidney transplant, the method comprising: administering rejection therapy to a patient determined to have: a urinary protein level of CXCL9 greater than 78.832 pg/mL; a urinary protein level of CXCL10 greater than 8.5748 pg/mL; a urinary protein level of CCL2 greater than 205.67 pg/mL; and a urinary protein level of VEF-A greater than 142.98 pg/mL; and administering standard immunosuppression therapy to a patient determined not to have: a urinary protein level of CXCL9 greater than 78.832 pg/mL; a urinary protein level of CXCL10 greater than 8.5748 pg/mL; a urinary protein level of CCL2 greater than 205.67 pg/mL; and a urinary protein level of VEF-A greater than 142.98 pg/mL.
Paragraph 8: A method of treating kidney transplant rejection in a patient who has received a kidney transplant, the method comprising: administering rejection therapy to a patient determined to have: a urinary protein level of CXCL9 greater than a normal or borderline level in Table 1; a urinary protein level of CXCL10 greater than a normal or borderline level in Table 1; a urinary protein level of CCL2 greater than a normal or borderline level in Table 1; and a urinary protein level of VEF-A greater than a normal or borderline level in Table 1; and administering standard immunosuppression therapy to a patient determined not to have: a urinary protein level of CXCL9 greater than a normal or borderline level in Table 1; a urinary protein level of CXCL10 greater than a normal or borderline level in Table 1; a urinary protein level of CCL2 greater than a normal or borderline level in Table 1; and a urinary protein level of VEF-A greater than a normal or borderline level in Table 1.
Paragraph 9: The method of paragraph 8, wherein the normal or borderline level in Table 1 is the 75% tile normal or borderline level.
Paragraph 10: The method of any of the preceding paragraphs, wherein the rejection therapy comprises a test to identify disease in the graft, a renal biopsy, methylprednisolone, plasma exchange, intravenous immunoglobulin, anti-thymocyte globulin, anti-CD20 antibody, lymphocyte-depleting antibody, or a combination thereof.
Paragraph 11: The method of any of the preceding paragraphs, wherein the rejection therapy comprises methylprednisolone, plasma exchange, intravenous immunoglobulin, anti-thymocyte globulin, anti-CD20 antibody, lymphocyte-depleting antibody, or a combination thereof.
Paragraph 12: The method of any of the preceding paragraphs, wherein the anti-CD20 antibody is rituximab, ocrelizumab, ofatumumab, or obinutuzumab.
Paragraph 13: The method of any of the preceding paragraphs, wherein the lymphocyte-depleting antibody is muromonab.
Paragraph 14: The method of any of the preceding paragraphs, wherein the standard immunosuppression therapy comprises a reduction or cessation of a corticosteroid, calcineurin inhibitor, mTOR inhibitor, belatacept, everolimus, prednisone, tacrolimus, mycophenolate, prednisolone, cyclosporine, siroliums, or azathioprine; or an avoidance of a further test or biopsy.
Paragraph 15: The method of any of the preceding paragraphs, wherein the standard immunosuppression therapy comprises corticosteroids, calcineurin inhibitors, mTOR inhibitors, or a combination thereof.
Paragraph 16: The method of any of the preceding paragraphs, wherein the standard immunosuppression therapy comprises belatacept, everolimus, prednisone, tacrolimus, mycophenolate, prednisolone, cyclosporine, siroliums, azathioprine, or a combination thereof.
Paragraph 17: A method of detecting kidney tissue, optionally kidney tissue from a kidney transplant, undergoing pathological apoptosis, the method comprising: contacting a urine sample obtained from the kidney tissue with antibodies specific for each of CXCL9, CXCL10, CCL2, and VEGF-A proteins; detecting the amount of each of CXCL9, CXCL10, CCL2, and VEGF-A bound to the antibodies, providing a data set comprising values for the level of each of CXCL9, CXCL10, CCL2, and VEGF-A detected.
Paragraph 18: The method of any one of the preceding paragraphs, wherein the detecting further comprises analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations.
Paragraph 19: The method of paragraph 18, wherein the weighting parameters are, 0.73797 for CXCL9, −0.046218 for CXCL10, 0.020161 for CCL2, and −0.21309 for VEGF-A.
Paragraph 20: The method of paragraph 19, wherein the logistical regression module further comprises applying a constant.
Paragraph 21: The method of paragraph 20, wherein the constant is −4.0624.
Paragraph 22: The method of paragraph 18, wherein the data set further comprises a value for the time from transplant of the kidney.
Paragraph 23: The method of paragraph 22, wherein the weighting process further comprises applying a weighting parameter for the time from transplant of the kidney.
Paragraph 24: The method of paragraph 23, wherein the weighting parameters are 0.75542 for CXCL9, 0.057341 for CXCL10, 0.095251 for CCL2, −0.34348 for VEGF-A, and 0.71722 for time from transplant.
Paragraph 25: The method of paragraph 24, wherein the weighting process further comprises applying a constant.
Paragraph 26: The method of paragraph 25, wherein the constant is −7.1573.
Paragraph 27: The method of any one of the preceding paragraphs, further comprising: determining a score based on the urinary protein levels; and identifying the kidney tissue as undergoing apoptosis based on the score being above a predetermined threshold.
Paragraph 28: The method of any one of the preceding paragraphs, further comprising identifying whether the kidney tissue is undergoing apoptosis and outputting said identification on a display.
Paragraph 29: The method of any one of the preceding paragraphs, wherein the urine sample is obtained from a patient and the method further comprises assigning the patient to a rejection category according to the score and outputting the rejection category on a display.
Paragraph 30: The method of any of the preceding paragraphs, wherein the urinary protein levels are normalized to urinary creatinine.
Paragraph 31: The method of any of the preceding paragraphs, wherein the patient is a pediatric patient.
Paragraph 32: A method of treating kidney transplant rejection in a patient who has received a kidney transplant, the method comprising: determining a urinary protein level of CXCL9; a urinary protein level of CXCL10; a urinary protein level of CCL2; and a urinary protein level of VEGF-A of a urine sample obtained from the kidney transplant; predicting whether the patient will experience an acute rejection based on an acute rejection prediction score output from a logistic regression model, wherein the urinary protein level of CXCL9; the urinary protein level of CXCL10; the urinary protein level of CCL2; and the urinary protein level of VEF-A measured from the patient are input into the logistic regression model; administering rejection therapy to the patient if the acute rejection prediction score exceeds a predetermined threshold; and administering standard immunosuppression therapy to the patient if the acute rejection prediction score does not exceed a predetermined threshold.
Paragraph 33: The method of paragraph 32, further comprising determining a time after the transplant is received by the patient, and wherein the time after the transplant is input into the logistic regression model.
Paragraph 34: The method of any of the preceding paragraphs, wherein the logistic regression model includes a preliminary technical control step checking that the determined CXCL9 and CXCL10 levels are correlated to within a predetermined standard error (S.E.) value of a training set linear regression fit from one another with a predetermined slope and a predetermined y-intercept value.
Paragraph 35: The method of any of the preceding paragraphs, wherein the logistic regression model includes a −4.0624 constant 1, a weighting value of −0.020161 for the CCL2 level, a weighting value of −0.046218 for the CXCL10 level, a weighting value of 0.73797 for the CXCL9 level, and a weighting value of −0.21309 for the VEGF-A level.
Paragraph 36: The method of any of the preceding paragraphs, wherein the logistic regression model includes a −7.1573 constant 1, a weighting value of 0.095251 for the CCL2 level, a weighting value of 0.057341 for the CXCL10 level, a weighting value of 0.75542 for the CXCL9 level, a weighting value of −0.34348 for the VEGF-A level, and a weighting value of 0.71722 for the time after the transplant.
Paragraph 37: The method of any of the preceding paragraphs, wherein the urinary protein level of CXCL9; the urinary protein level of CXCL10; the urinary protein level of CCL2; the urinary protein level of VEF-A; and time from transplant are modified by adding a +1 pseudocount unit and by log 2-transforming the resulting values.
Paragraph 38: The method of any of paragraphs 7-37, wherein the contacting and/or detecting steps are performed in an automated device.
Paragraph 39: The method of paragraph 38, wherein the automated device comprises a cartridge comprising the antibodies.
Paragraph 40: The method of any of paragraphs 7-39, wherein the detecting step comprises simultaneously detecting the amount of each of CXCL9, CXCL10, CCL2, and VEGF-A bound to the antibodies.
Paragraph 41: The method of any of paragraphs 7-40, wherein the urinary protein levels are normalized to urinary creatinine.
Paragraph 42: A cartridge comprising antibodies specific for each of the CXCL9, CXCL10, CCL2, and VEGF-A proteins, for use in the method of any of paragraphs 1-41.
Paragraph 43: A method of diagnosing kidney rejection in a subject who has received a kidney transplant, the method comprising,
   a) measuring the combination of:
   a urinary protein level of CXCL9;
   a urinary protein level of CXCL10;
   a urinary protein level of CCL2; and
   a urinary protein level of VEGF-A
in a urine sample obtained from the subject;
wherein the measuring is performed using a BIOTECHNE cartridge comprising detection antibodies for CXCL9, CXCL10, CCL2, and VEGF-A; and
b) determining an acute rejection prediction score output from a logistic regression model using the levels measured in step a); and
determining the subject has kidney rejection the acute rejection prediction score exceeds a predetermined threshold.
Paragraph 44: The method of paragraph 43, wherein the logistic regression model further comprises the time elapsed from the kidney transplant.
Paragraph 45: The method of any one of paragraphs 43-44, wherein the logistic regression model includes a preliminary technical control step checking that the determined CXCL9 and CXCL10 levels are correlated to within a predetermined standard error (S.E.) value of a training set linear regression fit from one another with a predetermined slope and a predetermined y-intercept value.
Paragraph 46: The method of any one of paragraphs 43-45, wherein the logistic regression model includes a −4.0624 constant 1, a weighting value of −0.020161 for the CCL2 level, a weighting value of −0.046218 for the CXCL10 level, a weighting value of 0.73797 for the CXCL9 level, and a weighting value of −0.21309 for the VEGF-A level.
Paragraph 47: The method of any one of paragraphs 43-45, wherein the logistic regression model includes a −7.1573 constant 1, a weighting value of 0.095251 for the CCL2 level, a weighting value of 0.057341 for the CXCL10 level, a weighting value of 0.75542 for the CXCL9 level, a weighting value of −0.34348 for the VEGF-A level, and a weighting value of 0.71722 for the time after the transplant.
Paragraph 48: The method of any one of paragraphs 43-47, wherein the urinary protein level of CXCL9; the urinary protein level of CXCL10; the urinary protein level of CCL2; the urinary protein level of VEGF-A; and time from transplant are modified by adding a +1 pseudocount unit and by log 2-transforming the resulting values.
Paragraph 49: The method of any one of paragraphs 43-48, wherein the measuring is performed in an ELLA device.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Example 1: Use of the ProteinSimple/Ella Platform to Develop a Urine Specific Assay for the Sensitive Monitoring of Renal Transplant Patients at Point-of-Care

The inventors designed and utilized a “cart” from BioTechne (for ProteinSimple's platform permitting multiprotein assays to be performed in an automated manner) that measures CCL2, CXCL9, CXCL10 and VEGFA. These markers have not been used in combination or advanced into clinical care as: 1) established technology does not allow for the use of reliable cut-off levels for normal/stable vs. disease, 2) the technology for the assay cannot be cross validated among different clinical sites and 3) prior mutli-plex/multitasking assays are not reliable for urine biomarker assays. Using this technology and cart assay, all 4 molecules are measured together, and the inventors developed an algorithm based on the assay to evaluate cut-off levels for use in determining if a renal transplant patient is either stable or at risk of disease.

Pilot studies were performed in urine from 517 renal transplant patients where urine was collected at the same time as a renal biopsy. Correlations with normal vs disease were performed. Cut off levels are attached in the Table 1. Subsequently, the inventors developed an algorithm to establish the utility of this 4-molecule assay. This assay is called score4 (patent-able) and score4+T (in which time post-transplant is taken into consideration) and we noted that the assay is reliable within a 14-day period of an event noted on a renal biopsy. The assay is most reliable to identify stable patients and it can be adapted for use in diseased patients alone, but can be optimized further if urine creatinine is assessed. See Tables 2 and 3. The score4 assay has been validated this assay in two additional cohorts.
Purpose: Current clinical monitoring of renal transplant patients use serum creatinine as a measure of disease. However, recent studies indicate that 25-36% of clinically stable pediatric renal transplant recipients (using serum creatinine alone) will have rejection detected on surveillance renal biopsy. These findings highlight a critical need to advance care by implementing urinary biomarker profiling in the clinic to identify low-and high-risk patients. In this manner it will be possible to optimize immunosuppression and promote long-term graft survival.
Methods: The inventors tested a novel automated platform (ProteinSimple Inc.) by developing a unique cart (called a “transplant cart”) that can measure CXCL9, CXCL10, CCL2 and VEGF-A simultaneously in urine with high precision. The inventors tested the diagnostic utility in a training set of biorepository samples collected at the time of surveillance or indication renal biopsy from adult and pediatric renal transplant recipients. Levels were evaluated alone or normalized to urinary creatinine (UCr) and correlated with clinical histology as assessed in four centers. Non-parametric rank sum analysis and area under ROC curve (AUC) were used to determine diagnostic performance. Risk algorithms using combinations of all four biomarkers were validated in two additional cohorts and the platform assay was cross-validated in three laboratories. Using AUC as a indication of performance (Tables 2A-2B), it was noted that the transplant cart assay alone without use of UCr provides for outstanding performance.
Results: 517 training samples were assessed vs. histological diagnosis as normal (n=330), acute rejection (n=92) or border-line changes (n=95). Each biomarker independently discriminated normal vs. acute rejection (P<1×10−10), whereas only CCL2 trended higher with borderline changes (P=0.008); CXCL9 and CXCL10 provide the best performance for the diagnosis of acute rejection (AUC 0.85 and 0.82, respectively). An algorithm utilizing the performance of all four biomarkers (‘score 4’) was developed and it was found to have excellent diagnostic performance for acute rejection (AUC 0.86) outperforming any individual biomarker. Also, there was an almost perfect cross-correlation among CXCL9 and CXCL10 (rank, r=0.86; regression slope=0.98) to identify risk. Pediatric urine samples collected from two independent validation cohorts of clinically stable patients (n=52 and n=120) were simultaneously evaluated in three laboratories with almost perfect inter-operator reproducibility. In these validation cohorts, score 4 retained excellent performance for acute rejection at the 75% ile specificity. In contrast, score 4 had limited performance in borderline rejection but cut off levels have potential to identify risk for disease. Furthermore, the assay fails to establish disease in patients with BKV viremia (predominantly within 90 days of diagnosis) but it is efficient in the evaluation of normal, low risk patients with BKV viremia.

It is also noted that cut offs levels for low risk normal stable patients decrease over time and that the discrimination between stable, borderline rejection (by Banff classification pre and post 2019) and acute rejection is more notable at times >1year post transplant. Trends over time of all four biomarkers (score4) distinguish normal stable patients from those with either borderline or acute rejection. Score4+T (time) was optimal to identify stability of renal transplant patients in the long term.
Discussion: This study represents one of the largest cohort analyses to date, and demonstrates that the integration of urinary biomarkers into pediatric point-of-care can optimize outcomes.

Algorithm Development: The inventors developed two logistic regression models (denoted score.4 and score.4+T) for identifying stable low risk patients following renal transplantation and to identify those at risk for disease (including acute rejection) using specific linear combinations of concentration levels of 4 urinary biomarkers (CCL2, CXCL9, CXCL10, VEGF-A) and the time from transplant (in months). The models include a common preliminary technical control step to check that CXCL9 and CXCL10 levels are correlated to within 10 standard error (S.E.) of a specific training set linear regression fit from one another with slope 0.98656 (S.E. 0.026078) and y-intercept 1.9229 (S.E. 0.11403). The specific linear combination for each mod-el is as follows: for score.4, −4.0624 (constant 1), −0.020161 (CCL2), −0.046218 (CXCL10), 0.73797 (CXCL9), −0.21309 (VEGF-A); and for score.4+T, −7.1573 (constant 1), 0.095251 (CCL2), 0.057341 (CXCL10), 0.75542 (CXCL9), −0.34348 (VEGF-A), 0.71722 (time-from-transplant), where biomarker quantity and time from transplant are respectively modified by adding 1 pseudocount unit and then log 2-transformed. The output from each model is a score that quantifies the likelihood of acute rejection in a patient with their inputted biomarker quantities and time-from-transplant.

TABLE 1
Maximum, minimum, interquartile range (IQR) for respective biomarker
concentrations and score4 stratified by age group.

| | CCL2 (pg/ml) | | | CXCL9 (pg/ml) | | | CXCL10 (pg/ml) | |
|---|---|---|---|---|---|---|---|---|
| All ages | | | | | | | | |
| Diagnosis (n) | N (330) | B (95) | AR (92) | N (330) | B (95) | AR (92) | N (330) | B (95) |
| 25% ile | 131.24 | 171.78 | 244.43 | 14.829 | 14.143 | 93.907 | 3.1 | 2.929 |
| 50% ile | 238.94 | 326.9 | 552.19 | 35.15 | 36.766 | 260.96 | 7.7015 | 9.616 |
| 75% ile | 452.31* | 705.43 | 922.26 | 84.13* | 102.59 | 807.95 | 22.136* | 27.267 |
| <18 yrs of age | | | | | | | | |
| Diagnosis (n) | N (125) | B (64) | AR (35) | N (125) | B (64) | AR (35) | N (125) | B (64) |
| 25% ile | 132.29 | 183.41 | 205.67 | 7.8733 | 13.027 | 78.832 | 2.5405 | 2.966 |
| 50% ile | 263.42 | 399.11 | 539.75 | 23.739 | 32.544 | 154.1 | 6.887 | 10.67 |
| 75% ile | 555.25* | 749.68 | 782.07 | 52.299* | 112.29 | 262.84 | 21.425* | 38.171 |
| ≥18 yrs of age | | | | | | | | |
| Diagnosis (n) | N (205) | B (31) | AR (56) | N (205) | B (31) | AR (56) | N (205) | B (31) |
| 25% ile | 126.38 | 144.37 | 315.12 | 17.884 | 21.297 | 102.79 | 3.4638 | 2.9093 |
| 50% ile | 216 | 271.6 | 565.99 | 47.947 | 38.548 | 468.95 | 8.821 | 8.135 |
| 75% ile | 431.31* | 411.57 | 1157.1 | 105.64* | 102.08 | 1240.7 | 22.35* | 21.278 |

| | CXCL10 (pg/ml) | VEGF-A (pg/ml) | | | Score4 | | |
|---|---|---|---|---|---|---|---|
| All ages | | | | | | | |
| Diagnosis (n) | AR (92) | N (330) | B (95) | AR (92) | N (330 | B (95) | AR (92) |
| 25% ile | 12.667 | 94.258 | 107.36 | 179.8 | −2.8504 | −2.9172 | −1.2962 |
| 50% ile | 52.332 | 165.89 | 188.46 | 281.62 | −2.1798 | −2.0571 | −0.4062 |
| 75% ile | 173.05 | 333.01* | 308.4 | 480.79 | −1.3514 | −1.1599 | 0.7171 |
| <18 yrs of age | | | | | | | |
| Diagnosis (n) | AR (35) | N (125) | B (64) | AR (35) | N (125) | B (64) | AR (35) |
| 25% ile | 8.5748 | 100.21 | 83.569 | 142.98 | −3.2465 | −3.0227 | −1.5176 |
| 50% ile | 28.538 | 165 | 178.01 | 267.51 | −2.5018 | −2.1197 | −0.7531 |
| 75% ile | 51.727 | 333.26* | 287.83 | 416.35 | −1.9191 | −1.1887 | −0.4050 |
| ≥18 yrs of age | | | | | | | |
| Diagnosis (n) | AR (56) | N (205) | B (31) | AR (56) | N (205) | B (31) | AR (56) |
| 25% ile | 24.16 | 92.658 | 136.17 | 202.83 | −2.6318 | −2.6366 | −1.1726 |
| 50% ile | 87.251 | 166.75 | 205.27 | 286.43 | −1.9708 | −1.9737 | 0.1685 |
| 75% ile | 284.25 | 331.97* | 352.23 | 561.96 | −1.1116 | −1.1547 | 1.173 |

*75% specificity threshold was used in analyses as a high threshold for biomarker levels.
Age (at transplant) was not available for every patient; those without age were excluded from levels identified on patients listed as < or >18 yrs age but are included in the ‘all ages’ group
N = normal;
B = Borderline rejection;
AR = acute rejection

TABLE 2A
The area under the empirical receiver operating characteristic (AUROC) curve and their 95% confidence intervals for
logistic regression models of normal versus acute rejection using the 4 biomarkers individually and together (Score4)

| | Number | CCL2 | CXCL10 | CXCL9 | VEGF-A | Score4 |
|---|---|---|---|---|---|---|
| Patients | (N/AR) | AUROC [95% CI] | AUROC [95% CI] | AUROC [95% CI] | AUROC [95% CI] | AUROC [95% CI] |
| All ages | 330/92 | 0.6919 | 0.7833 | 0.8278 | 0.6579 | 0.8303 |
| | | [0.6409, 0.7428] | [0.7379, 0.8288] | [0.7861, 0.8695] | [0.6056, 0.7103] | [0.7889, 0.8717] |
| <18 yrs of age | 125/35 | 0.6158 | 0.7065 | 0.8341 | 0.6286 | 0.8551 |
| | | [0.5284, 0.7032] | [0.6247, 0.7883] | [0.7672, 0.9009] | [0.5418, 0.7154] | [0.7918, 0.9183] |
| ≥18 yrs of age | 205/56 | 0.7355 | 0.8281 | 0.8358 | 0.6799 | 0.8309 |
| | | [0.6735, 0.7974] | [0.7751, 0.8810] | [0.7838, 0.8879] | [0.6144, 0.7454] | [0.7783, 0.8836] |
| All ages | 317/84 | 0.7275 | 0.8217 | 0.8500 | 0.7169 | 0.8497 |
| Ratio/Creatinine | | [0.6771, 0.7779] | [0.7784, 0.8651] | [0.8095, 0.8904] | [0.6659, 0.7679] | [0.8093, 0.8902] |
| <18 yrs of age | 121/33 | 0.6018 | 0.7383 | 0.8500 | 0.6101 | 0.8512 |
| Ratio/Creatinine | | [0.5121, 0.6915] | [0.6578, 0.8188] | [0.7846, 0.9154] | [0.5207, 0.6994] | [0.7861, 0.9164] |
| ≥18 yrs of age | 196/50 | 0.7872 | 0.8658 | 0.8610 | 0.7654 | 0.8602 |
| Ratio/Creatinine | | [0.7280, 0.8465] | [0.8165, 0.9151] | [0.8110, 0.9111] | [0.7041, 0.8267] | [0.8100, 0.9104] |
| All ages | 275/84 | 0.6951 | 0.7946 | 0.8477 | 0.6473 | 0.8570 |
| <14 day Pre/Post Biopsy | | [0.6400, 0.7502] | [0.7462, 0.8429] | [0.8047, 0.8907] | [0.5901, 0.7045] | [0.8151, 0.8989] |
| <18 yrs of age | 116/33 | 0.6068 | 0.7187 | 0.8550 | 0.6259 | 0.8796 |
| <14 day Pre/Post Biopsy | | [0.5159, 0.6978] | [0.6349, 0.8024] | [0.7894, 0.9206] | [0.5358, 0.7160] | [0.8190, 0.9402] |
| ≥18 yrs of age | 159/50 | 0.7523 | 0.8429 | 0.8559 | 0.6647 | 0.8577 |
| <14 day Pre/Post Biopsy | | [0.6845, 0.8201] | [0.7857, 0.9000] | [0.8008, 0.9111] | [0.5905, 0.7388] | [0.8029, 0.9126] |
| All ages | 264/78 | 0.7325 | 0.8369 | 0.8699 | 0.7177 | 0.8711 |
| Ratio/Creatinine, and | | [0.6782, 0.7868] | [0.7916, 0.8822] | [0.8286, 0.9111] | [0.6625, 0.7729] | [0.8300, 0.9122] |
| <14 day Pre/Post Biopsy | | | | | | |
| <18 yrs of age | 112/32 | 0.5910 | 0.7567 | 0.8717 | 0.6127 | 0.8733 |
| Ratio/Creatinine, and | | [0.4978, 0.6841] | [0.6754, 0.8380] | [0.8083, 0.9350] | [0.5204, 0.7050] | [0.8103, 0.9364] |
| <14 day Pre/Post Biopsy | | | | | | |
| ≥18 yrs of age | 152/45 | 0.8066 | 0.8836 | 0.8803 | 0.7709 | 0.8807 |
| Ratio/Creatinine, and | | [0.7427, 0.8705] | [0.8317, 0.9355] | [0.8277, 0.9328] | [0.7029, 0.8389] | [0.8283, 0.9332] |
| <14 day Pre/Post Biopsy | | | | | | |

TABLE 2B
The area under the empirical receiver operating characteristic (AUROC) curve and their 95% confidence intervals for logistic
regression models of normal versus acute rejection using the 4 biomarkers with adjustment for the time from transplant.

| | Number | CCL2 + T | CXCL10 + T | CXCL9 + T | VEGF-A + T | Score4 + T |
|---|---|---|---|---|---|---|
| Patients | (N/AR) | AUROC [95% CI] | AUROC [95% CI] | AUROC [95% CI] | AUROC [95% CI] | AUROC [95% CI] |
| All ages | 326/91 | 0.7924 | 0.8446 | 0.8725 | 0.7558 | 0.8790 |
| | | [0.7474, 0.8374] | [0.8044, 0.8848] | [0.8355, 0.9095] | [0.7081, 0.8034] | [0.8428, 0.9152] |
| <18 yrs of age | 124/35 | 0.8318 | 0.8472 | 0.9039 | 0.8062 | 0.9097 |
| | | [0.7644, 0.8992] | [0.7824, 0.9121] | [0.8508, 0.9570] | [0.7350, 0.8775] | [0.8580, 0.9613] |
| ≥18 yrs of age | 202/56 | 0.7805 | 0.8495 | 0.8528 | 0.7273 | 0.8626 |
| | | [0.7220, 0.8390] | [0.7990, 0.9001] | [0.8028, 0.9029] | [0.6644, 0.7902] | [0.8140, 0.9113] |
| All ages | 313/83 | 0.8244 | 0.8774 | 0.8922 | 0.7966 | 0.8995 |
| Ratio/Creatinine | | [0.7810, 0.8677] | [0.8400, 0.9148] | [0.8569, 0.9275] | [0.7508, 0.8425] | [0.8652, 0.9337] |
| <18 yrs of age | 120/33 | 0.8429 | 0.8717 | 0.9215 | 0.8076 | 0.9189 |
| Ratio/Creatinine | | [0.7761, 0.9098] | [0.8103, 0.9332] | [0.8720, 0.9709] | [0.7351, 0.8800] | [0.8688, 0.9691] |
| ≥18 yrs of age | 193/50 | 0.8302 | 0.8856 | 0.8751 | 0.7950 | 0.8879 |
| Ratio/Creatinine | | [0.7755, 0.8848] | [0.8393, 0.9319] | [0.8270, 0.9233] | [0.7363, 0.8538] | [0.8419, 0.9338] |
| All ages | 273/83 | 0.8071 | 0.8671 | 0.8986 | 0.7536 | 0.9131 |
| <14 day Pre/Post | | [0.7596, 0.8545] | [0.8263, 0.9079] | [0.8624, 0.9349] | [0.7018, 0.8054] | [0.8793, 0.9470] |
| Biopsy | | | | | | |
| <18 yrs of age | 115/33 | 0.8408 | 0.8748 | 0.9289 | 0.8148 | 0.9428 |
| <14 day Pre/Post | | [0.7725, 0.9092] | [0.8130, 0.9367] | [0.8808, 0.9769] | [0.7422, 0.8874] | [0.8994, 0.9862] |
| Biopsy | | | | | | |
| ≥18 yrs of age | 158/50 | 0.7995 | 0.8696 | 0.8770 | 0.7181 | 0.8948 |
| <14 day Pre/Post | | [0.7365, 0.8625] | [0.8166, 0.9226] | [0.8253, 0.9287] | [0.6473, 0.7889] | [0.8465, 0.9431] |
| Biopsy | | | | | | |
| All ages | 262/77 | 0.8397 | 0.9001 | 0.9159 | 0.7996 | 0.9257 |
| Ratio/Creatinine, and | | [0.7945, 0.8848] | [0.8631, 0.9370] | [0.8818, 0.9501] | [0.7503, 0.8489] | [0.8933, 0.9580] |
| <14 day Pre/Post | | | | | | |
| Biopsy | | | | | | |
| <18 yrs of age | 111/32 | 0.8502 | 0.8933 | 0.9445 | 0.8125 | 0.9434 |
| Ratio/Creatinine, and | | [0.7824, 0.9181] | [0.8346, 0.9520] | [0.9010, 0.9881] | [0.7383, 0.8867] | [0.8995, 0.9874] |
| <14 day Pre/Post | | | | | | |
| Biopsy | | | | | | |
| ≥18 yrs of age | 151/45 | 0.8548 | 0.9080 | 0.8971 | 0.7996 | 0.9148 |
| Ratio/Creatinine, and | | [0.7976, 0.9119] | [0.8611, 0.9549] | [0.8479, 0.9464] | [0.7346, 0.8645] | [0.8695, 0.9601] |
| <14 day Pre/Post | | | | | | |
| Biopsy | | | | | | |

1. A method of identifying a kidney transplant patient as being stable and not in transplant rejection, the method comprising:
a) contacting a urine sample with antibodies specific for each of the CXCL9, CXCL10, CCL2, and VEGF-A proteins;
b) detecting the amount of each of CXCL9, CXCL10, CCL2, and VEGF-A bound to the antibodies, providing a data set comprising values for the level of each of CXCL9, CXCL10, CCL2, and VEGF-A detected;
c) analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant;
wherein the weighting parameters are, 0.73797 for CXCL9, −0.046218 for CXCL10, 0.020161 for CCL2, and −0.21309 for VEGF-A and the constant is −4.0624; thereby providing a score; and
d) identifying a kidney transplant patient as being stable and not in transplant rejection based on the score being below a predetermined threshold.

2. A method of identifying a kidney transplant patient as being stable and not in transplant rejection, the method comprising:
a) contacting a urine sample with antibodies specific for each of CXCL9, CXCL10, CCL2, and VEGF-A proteins;
b) detecting the amount of each of CXCL9, CXCL10, CCL2, and VEGF-A bound to the antibodies, providing a data set comprising values for the level of each of CXCL9, CXCL10, CCL2, and VEGF-A detected;
c) analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations, and the logistical regression module further comprises applying a constant; and
d) ional change in each of CXCL9, CXCL10, CCL2, and VEGF-A between normal and acute rejection populations, and the logistical regression module further comprises applying a constant; and
the data set further comprises a value for the time from transplant of the kidney and the weighting process further comprises applying a weighting parameter for the time from transplant of the kidney,
wherein the weighting parameters are 0.75542 for CXCL9, 0.057341 for CXCL10, 0.095251 for CCL2, −0.34348 for VEGF-A, and 0.71722 for time from transplant; and
wherein the constant is −7.1573 thereby providing a score; and
e) identifying a kidney transplant patient as being stable and not in transplant rejection based on the score being below a predetermined threshold.

3.-6. (canceled)

7. A method of treating kidney transplant rejection in a patient who has received a kidney transplant, the method comprising:
a) administering rejection therapy to a patient determined to have:
i) a urinary protein level of CXCL9 greater than 78.832 pg/mL;
ii) a urinary protein level of CXCL10 greater than 8.5748 pg/mL;
iii) a urinary protein level of CCL2 greater than 205.67 pg/mL; and
iv) a urinary protein level of VEGF-A greater than 142.98 pg/mL; and
b) administering standard immunosuppression therapy to a patient determined not to have:
i) a urinary protein level of CXCL9 greater than 78.832 pg/mL;
ii) a urinary protein level of CXCL10 greater than 8.5748 pg/mL;
iii) a urinary protein level of CCL2 greater than 205.67 pg/mL; and
iv) a urinary protein level of VEGF-A greater than 142.98 pg/mL.

8. A method of treating kidney transplant rejection in a patient who has received a kidney transplant, the method comprising:
a) administering rejection therapy to a patient determined to have:
i) a urinary protein level of CXCL9 greater than a normal or borderline level in Table 1;
ii) a urinary protein level of CXCL10 greater than a normal or borderline level in Table 1;
iii) a urinary protein level of CCL2 greater than a normal or borderline level in Table 1; and
iv) a urinary protein level of VEGF-A greater than a normal or borderline level in Table 1; and
b) administering standard immunosuppression therapy to a patient determined not to have:
i) a urinary protein level of CXCL9 greater than a normal or borderline level in Table 1;
ii) a urinary protein level of CXCL10 greater than a normal or borderline level in Table 1;
iii) a urinary protein level of CCL2 greater than a normal or borderline level in Table 1; and
iv) a urinary protein level of VEGF-A greater than a normal or borderline level in Table 1.

9. (canceled)

10. The method of claim 7, wherein the rejection therapy comprises a test to identify disease in the graft, a renal biopsy, methylprednisolone, plasma exchange, intravenous immunoglobulin, anti-thymocyte globulin, anti-CD20 antibody, lymphocyte-depleting antibody, or a combination thereof.

11. The method of claim 7, wherein the rejection therapy comprises methylprednisolone, plasma exchange, intravenous immunoglobulin, anti-thymocyte globulin, anti-CD20 antibody, lymphocyte-depleting antibody, or a combination thereof.

12. The method of claim 7, wherein the anti-CD20 antibody is rituximab, ocrelizumab, ofatumumab, or obinutuzumab or wherein the lymphocyte-depleting antibody is muromonab.

13. (canceled)

14. The method of claim 7, wherein the standard immunosuppression therapy comprises a reduction or cessation of a corticosteroid, calcineurin inhibitor, mTOR inhibitor, belatacept, everolimus, prednisone, tacrolimus, mycophenolate, prednisolone, cyclosporine, siroliums, or azathioprine; or an avoidance of a further test or biopsy.

15. The method of claim 7, wherein the standard immunosuppression therapy comprises corticosteroids, calcineurin inhibitors, mTOR inhibitors, or a combination thereof.

16. The method of claim 7, wherein the standard immunosuppression therapy comprises belatacept, everolimus, prednisone, tacrolimus, mycophenolate, prednisolone, cyclosporine, siroliums, azathioprine, or a combination thereof.

17. A method of detecting kidney tissue, optionally kidney tissue from a kidney transplant, undergoing pathological apoptosis, the method comprising:
a) contacting a urine sample obtained from the kidney tissue with antibodies specific for each of CXCL9, CXCL10, CCL2, and VEGF-A proteins;
b) detecting the amount of each of CXCL9, CXCL10, CCL2, and VEGF-A bound to the antibodies, providing a data set comprising values for the level of each of CXCL9, CXCL10, CCL2, and VEGF-A detected.

18. The method of claim 7, wherein the detecting further comprises analyzing the data set with a control system comprising one or more processors, the control system configured to execute machine executable code executing a logistical regression model applying a weighting parameter for each of CXCL9, CXCL10, CCL2, and VEGF-A, wherein each weighting parameter identifies the proportional change in each of CXCL9, CXCL10, CCL2, and VEGF-A between normal and acute rejection populations.

19. The method of claim 18, wherein the weighting parameters are, 0.73797 for CXCL9, −0.046218 for CXCL10, 0.020161 for CCL2, and −0.21309 for VEGF-A.

20. (canceled)

21. (canceled)

22. The method of claim 18, wherein the data set further comprises a value for the time from transplant of the kidney,
wherein the weighting process further comprises applying a weighting parameter for the time from transplant of the kidney, and
wherein the weighting parameters are 0.75542 for CXCL9, 0.057341 for CXCL10, 0.095251 for CCL2, −0.34348 for VEGF-A, and 0.71722 for time from transplant.

23.-31. (canceled)

32. A method of treating kidney transplant rejection in a patient who has received a kidney transplant, the method comprising:
determining a urinary protein level of CXCL9; a urinary protein level of CXCL10; a urinary protein level of CCL2; and a urinary protein level of VEGF-A of a urine sample obtained from the kidney transplant;
predicting whether the patient will experience an acute rejection based on an acute rejection prediction score output from a logistic regression model, wherein the urinary protein level of CXCL9; the urinary protein level of CXCL10; the urinary protein level of CCL2; and the urinary protein level of VEGF-A measured from the patient are input into the logistic regression model;
administering rejection therapy to the patient if the acute rejection prediction score exceeds a predetermined threshold; and
administering standard immunosuppression therapy to the patient if the acute rejection prediction score does not exceed a predetermined threshold.

33.-36. (canceled)

37. The method of any of the preceding claim 7, wherein the urinary protein level of CXCL9; the urinary protein level of CXCL10; the urinary protein level of CCL2; the urinary protein level of VEF-A; and time from transplant are modified by adding a +1 pseudocount unit and by log 2-transforming the resulting values.

38. (canceled)

39. (canceled)

40. The method of any of claim 7, wherein the detecting step comprises simultaneously detecting the amount of each of CXCL9, CXCL10, CCL2, and VEGF-A bound to the antibodies.

41. The method of any of claim 7, wherein the urinary protein levels are normalized to urinary creatinine.

42. A cartridge comprising antibodies specific for each of the CXCL9, CXCL10, CCL2, and VEGF-A proteins, for use in the method of any of claim 1.

43. A method of diagnosing kidney rejection in a subject who has received a kidney transplant, the method comprising,
a. measuring the combination of:
a urinary protein level of CXCL9;
a urinary protein level of CXCL10;
a urinary protein level of CCL2; and
a urinary protein level of VEGF-A
in a urine sample obtained from the subject;
wherein the measuring is performed using a BIOTECHNE cartridge comprising detection antibodies for CXCL9, CXCL10, CCL2, and VEGF-A; and
b. determining an acute rejection prediction score output from a logistic regression model using the levels measured in step a); and
determining the subject has kidney rejection the acute rejection prediction score exceeds a predetermined threshold.

44.-49. (canceled)
